(19) United States
(12) Patent Application Publication
(10) Pub. No.: US 2008/0076145 A1
(43) Pub. Date: Mar. 27, 2008

(54) Transgenic flies expressing tau and amyloid precursor fragment

(75) Inventors: Christopher Cummings, San Francisco, CA (US); Matthew B. Mahoney, Newton, MA (US); Carol M. Singh, Somerville, MA (US)

Correspondence Address: PALMER & DODGE, LLP;KATHLEEN M. WILLIAMS, 111 HUNTINGTON AVENUE, BOSTON, MA, 02199, US

(73) Assignee: En Vivo Pharmaceuticals, Inc.

(21) Appl. No.: 11/818,413

(22) Filed: Jun. 14, 2007

Related U.S. Application Data
(60) Provisional application No. 60/814,227, filed on Jun. 16, 2006.
(60) Provisional application No. 60/815,986, filed on Jun. 23, 2006.

(51) Int. Cl. A01K 67/00; C12N 5/06; C12Q 1/20; G01N 33/00

(52) U.S. Cl. 435/29; 435/348; 800/12; 800/13; 800/3

(57) ABSTRACT

The present invention discloses a transgenic fly that expresses a carboxy terminal fragment of the human amyloid-β precursor protein (APP) and a double transgenic fly that expresses both the fragment of APP and tau protein. The transgenic flies of the present invention provide for models of neurodegenerative disorders, such as Alzheimer's disease. The invention further discloses methods for identifying genetic modifiers, as well as screening methods to identify therapeutic compounds to treat neurodegenerative disorders using the transgenic flies.

This application claims priority to Provisional Application Ser. Nos. 60/814,227, filed Jun. 16, 2006 and 60/815,986, filed Jun. 23, 2006, the contents of which are incorporated herein in their entirety.

### BACKGROUND

Alzheimer's disease (AD) is the most common neurodegenerative disorder in humans. The disease is characterized by a progressive impairment in cognition and memory. The hallmark of AD at the neuropathological level is the extracellular accumulation of the amyloid-β peptide (Aβ) in “senile” plaques, and the intracellular deposition of neurofibrillary tangles made of the microtubule-associated protein, tau. In neuronal tissue of AD patients, tau is hyperphosphorylated and adopts pathological conformations evident with conformation-dependent antibodies. The amyloid-β peptide is a cleavage product of the amyloid precursor protein (APP). In normal individuals, most of the Aβ is in a 40-amino acid form, but there are also minor amounts of Aβ that are 42 amino acids in length (Aβ42). In patients with AD there is an overabundance of Aβ42, which is thought to be the main toxic Aβ form.

A number of pathogenic mutations have been found within APP which are associated with hereditary forms of AD, several of which are located within the Aβ sequences. These mutations result in a phenotype different from AD, with massive amyloid accumulation in cerebral blood vessel walls. Two mutations, namely the Dutch (Glu22Gln) and the Flemish (Ala21Gly) mutations, have been reported (Levy, et al., Science 248, 1124-1126 (1990)), (van Broeckhoven et al. (1990)), (Hendriks, et al., Nature Genet. 1, 218-221 (1992)). Patients having these mutations suffer from cerebral hemorrhage and vascular symptoms. The vascular symptoms are caused by aggregation of Aβ in blood vessel walls (amyloid angiopathy). A third pathogenic intra-Aβ mutation was recently discovered in an Italian family (Glu22Lys), with clinical findings similar to the Dutch patients (Tagliavini, et al., Alz Report 2, S28 (1999)). Yet another pathogenic AD mutation within APP, the Arctic mutation (Glu22Gly), is also located within the Aβ peptide domain of the APP gene. Carriers of this mutation develop progressive dementia with clinical features typical of AD without symptoms of cerebrovascular disease. AD is distinctly characterized by accelerated formation of protofibrils comprising mutated Aβ peptides (Aβ40_{ARC} and/or Aβ42_{ARC}) compared to protofibril formation of wild type Aβ peptides. Finally, carriers of the Iowa mutation, carrying a Asp23Asn mutation within Aβ, exhibit severe cerebral amyloid angiopathy, widespread neurofibrillary tangles, and unusually extensive distribution of Aβ40 in plaques. (Grabowski et al., Ann. Neurol. 49: 691-693 (2001)).

Mutations of the APP gene outside the Aβ sequence have also been associated with Alzheimer's disease. These mutants encode amino acid substitutions in the C-terminal region of APP which affect cleavage by gamma secretase so as to increase the ratio of Aβ42 to Aβ40. Such mutations include the Austrian (Thr714Ile, codon numbering of APP770 isoform), Florida (Ile716Val), French (Val715Met), German (Val715Ala), Indiana (Val717Leu), and London (Val717Ile) mutations. See De Jonge et al., Hum. Molec. Gen. 10:1665-71 (2001).

A number of transgenic mouse models have been generated that express wild-type or mutant human APP. The mutant form of APP is differentially cleaved to result in increased amounts of Aβ42 deposited within Aβ plaques. These transgenic mice present with neurological symptoms of Alzheimer's disease, such as impaired memory and motor function (Janus C. et al., Curr. Neurol. Neurosci. Rep 1 (5): 451-457 (2001)). A transgenic mouse that expresses both mutant human APP and mutant human tau has also been generated (Jada, et. al., Science 293:1487-1491 (2001)). This double transgenic mouse is a rodent model for AD that shows enhanced neurofibrillary degeneration indicating that either APP or Aβ influences the formation of neurofibrillary tangles. While mouse models have proven very useful for testing potential AD therapeutics, their use for testing therapeutics is both expensive and time consuming. Thus, it would be beneficial to find alternative models, for example, non-mammalian models such as Caenorhabditis elegans or Drosophila melanogaster, which are less expensive and can be efficiently used to screen for therapeutic agents for Alzheimer's disease.

The use of Drosophila as a model organism has proven to be an important tool in the elucidation of human neurodegenerative pathways (reviewed in Fortini, M. and Bonini, N. Trends Genet. 16: 161-167 (2000)), as the Drosophila genome contains many relevant human orthologs that are extremely well conserved in function (Rubin, G. M., et al., Science 287: 2204-2215 (2000)). For example, Drosophila melanogaster carries a gene that is homologous to human APP which is involved in nervous system function. The gene, APP-like (APPL), is approximately 40% identical to APP695, the neuronal isoform (Rosen et al., Proc. Natl. Acad. Sci. U.S.A. 86:2478-2482 (1988)), and like human APP695 is exclusively expressed in the nervous system. Flies deficient for the APPL gene show behavioral defects which can be rescued by the human APP gene, suggesting that the two genes have similar functions in the two organisms (Luo et al., Neuron 9:595-605 (1992)). In addition, Drosophila models of polyglutamine repeat diseases (Jackson, G. R., et al., Neuron 21:633-642 (1998); Kazemi-Esfarani, P. and Benzer, S., Science 287:1837-1840 (2000); Femandez-Funez et al., Nature 408:101-6 (2000)), Parkinson's disease (Feany, M. B. and Bender, W. W., Nature 404:394-398 (2000)) and other diseases have been established which closely mimic the disease state in humans at the cellular and physiological levels, and have been successfully employed in identifying other genes that may be involved in these diseases. Thus, the power of Drosophila as a model system has been demonstrated in the ability to represent the disease state and to perform large scale genetic screens to identify critical components of disease.

### SUMMARY OF THE INVENTION

The present invention discloses transgenic flies that express a carboxy terminal fragment of human APP (CTFAPP), e.g., the C-terminal 99 or 100 amino acids, often referred to as “C99” and “C100” respectively. The somatic and germ cells of the transgenic flies of the invention comprise a transgene encoding CTFAPP, operatively linked to an expression control sequence. In some embodiments expression of the transgene results in the fly having an altered phenotype. In certain embodiments the altered phenotype is related to a form of neural degeneration or a predisposition thereto. In certain embodiments, the transgenic fly is Drosophila. The DNA sequence encoding CTFAPP may be fused to the DNA sequence for a signal peptide, e.g., via sequence for an amino acid linker. The transgene can be temporally or spatially regulated by the expression control sequence, which can be tissue-specific, time-specific or developmental stage-specific. In some embodiments, the CTFAPP is a mutant or variant form.

In some embodiments of the invention the transgenic fly comprises a second transgene, which encodes a tau protein. The second transgene is operatively linked to an expression control sequence. The double transgenic flies display a synergistic altered phenotype as compared to the altered phenotype displayed by transgenic flies expressing a form of CTFAPP alone. In some embodiments the tau protein is a human tau, for example one of the known splice variants of human tau.

Expression control sequences of the invention can be tissue-specific. In some embodiments the expression control sequence includes a UAS control element functionally coupled to a DNA sequence encoding GAL4. The GAL4 encoding sequence is driven by a tissue specific promoter or enhancer sequence. In certain embodiments the promoter or enhancer is specific for pan-neural expression or expression in brain or eye.

The invention also provides primary cell cultures obtained from a transgenic fly of the invention. Primary cell cultures can be used, for example, to identify agents active in neurodegenerative disease. A transgenic cell obtained from a transgenic fly can possess an altered phenotype related to a neurodegenerative disease such as Alzheimer's disease. The transgenic cell can have an altered phenotype, such as altered morphology or altered biochemical state of a molecular component, e.g., an altered phosphorylation state of a tau protein or an altered solubility of an amyloid polypeptide.

In another aspect, the invention relates to a method for identifying an agent active in neurodegenerative disease. The method comprises the steps of (1) contacting a candidate agent with a transgenic fly of the invention and (2) observing the phenotype of the transgenic fly, or a cell obtained from the transgenic fly, relative to a similar (control) transgenic fly or cell that has not been contacted with the candidate agent. An observable difference in the phenotype of the transgenic fly or cell that has been contacted with the candidate agent compared to the control fly or cell is indicative of an agent active in neurodegenerative disease.

The invention also relates to another method for identifying an agent active in neurodegenerative disease. The method comprises the steps of (1) contacting a candidate agent with a transgenic fly of the invention, or a cell obtained from such a fly, and to a wild type control fly or cell and (2) observing a difference in phenotype between the transgenic fly or cell and the control fly or cell, wherein a difference in phenotype is indicative of an agent active in neurodegenerative disease.

In a further aspect, the invention relates to a method of identifying a genetic modifier of the APP pathway or a gene which can affect Alzheimer's disease. The method comprises the steps of (1) crossing a transgenic fly comprising a transgene encoding CTFAPP, either wild type or one of the mutant forms listed above, and optionally comprising a transgene encoding a tau protein, with a fly whose genome comprises a mutation in a selected gene and (2) observing the progeny for alteration of a transgenic phenotype. Alteration of a phenotype associated with the transgene encoding a form of CTFAPP and/or tau indicates that the selected gene can modify the APP pathway or can affect Alzheimer's disease. The transgenes encoding a form of CTFAPP or tau are each operatively linked to a tissue-specific expression control sequence. The transgene encoding a form of CTFAPP is optionally fused to a signal sequence.

### BRIEF DESCRIPTION OF THE DRAWINGS

FIG. 1 shows an immunoblot demonstrating the presence of the Aβ peptide in transgenic flies expressing a CTFAPP. Details of the experiment are described in Example 1.

FIG. 2 depicts the declining locomotor ability of transgenic Drosophila as a function of age. Drosophila were subjected to a climbing assay as described in Example 3. The flies were either wild type (wt), or contained one transgene (tau or CTFAPP), or two transgenes (CTFAPP, tau).

### DETAILED DESCRIPTION OF THE INVENTION

The present invention discloses transgenic flies that express a human C-terminal APP fragment either alone or in combination with the tau protein. The transgenic flies exhibit neurodegeneration which can lead to a variety of altered phenotypes including locomotor phenotypes, behavioral phenotypes (e.g., appetite, mating behavior, and/or life span), and morphological phenotypes (e.g., shape, size, or location of a cell, organ, or appendage; or size, shape, or growth rate of the fly).

As used herein, the term “transgenic fly” refers to a fly whose somatic and germ cells contain a transgene operatively linked to a promoter, wherein the transgene encodes a human C-terminal APP fragment, and wherein the expression of said transgene in the nervous system results in said fly having a predisposition to, or resulting in, neural degeneration. The term “double transgenic fly” refers to a transgenic fly whose somatic and germ cells comprise at least two transgenes, wherein the transgenes encode the tau and human C-terminal APP fragment. Although the exemplified double transgenic fly is produced by crossing two single transgenic flies, the double transgenic fly of the present invention can be produced using any method known in the art for introducing foreign DNA into an animal. The terms “transgenic fly” and “double transgenic fly” include any developmental stages of the fly, i.e., embryonic, larval, pupal, and adult stages. The development of certain flies, e.g., Drosophila, is temperature dependent. The Drosophila egg is about a half millimeter long. It takes about one day after fertilization for the embryo to develop and hatch into a worm-like larva. The larva eats and grows continuously, molting one day, two days, and four days after hatching (first, second and third instars). After two days as a third instar larva, the larva molts one more time to form an immobile pupa. Over the next four days, the body is completely remodeled to give the adult winged form, which then hatches from the pupal case and is fertile after another day (timing of development is for 25° C.; at 18° C., development takes roughly twice as long).

As used herein, the term “neural degeneration” means a condition in the central nervous system that gives rise to morphologic, functional or developmental alteration of nervous or neurosensory organs, tissues, or cells; behavioral deficits; or locomotor deficits; wherein such alterations can be qualitatively or quantitatively analyzed in either larvae or adult flies.

As used herein, “fly” refers to a small insect with wings, especially a dipteran such as, for example, Drosophila. As used herein, the term “Drosophila” refers to any member of the Drosophilidae family, which include without limitation, Drosophila funebris, Drosophila multispina, Drosophila subfunebris, guttifera species group, Drosophila guttifera, Drosophila albomicans, Drosophila annulipes, Drosophila curviceps, Drosophila formosana, Drosophila hypocausta, Drosophila immigrans, Drosophila keplauana, Drosophila kohkoa, Drosophila nasuta, Drosophila neohypocausta, Drosophila niveifrons, Drosophila pallidiftons, Drosophila pulaua, Drosophila quadrilineata, Drosophila siamana, Drosophila sulfurigaster albostrigata, Drosophila sulfurigaster bilimbata, Drosophila sulfurigaster neonasuta, Drosophila Taxon F, Drosophila Taxon I, Drosophila ustulata, Drosophila melanica, Drosophila paramelanica, Drosophila tsigana, Drosophila daruma, Drosophila polychaeta, quinaria species group, Drosophila falleni, Drosophila nigromaculata, Drosophila palustris, Drosophila phalerata, Drosophila subpalustris, Drosophila eohydei, Drosophila hydei, Drosophila lacertosa, Drosophila robusta, Drosophila sordidula, Drosophila repletoides, Drosophila kanekoi, Drosophila virilis, Drosophila maculinatata, Drosophila ponera, Drosophila ananassae, Drosophila atripex, Drosophila bipectinata, Drosophila ercepeae, Drosophila malerkotliana malerkotliana, Drosophila malerkotliana pallens, Drosophila parabipectinata, Drosophila pseudoananassae pseudoananassae, Drosophila pseudoananassae nigrens, Drosophila varians, Drosophila elegans, Drosophila gunungcola, Drosophila eugracilis, Drosophila ficusphila, Drosophila erecta, Drosophila mauritiana, Drosophila melanogaster, Drosophila orena, Drosophila sechellia, Drosophila simulans, Drosophila teissieri, Drosophila yakuba, Drosophila auraria, Drosophila baimaii, Drosophila barbarae, Drosophila biauraria, Drosophila birchii, Drosophila bocki, Drosophila bocqueti, Drosophila burlai, Drosophila constricta (sensu Chen & Okada), Drosophila jambulina, Drosophila khaoyana, Drosophila kikkawai, Drosophila lacteicornis, Drosophila leontia, Drosophila lini, Drosophila mayri, Drosophila parvula, Drosophila pectinifera, Drosophila punjabiensis, Drosophila quadraria, Drosophila rufa, Drosophila seguyi, Drosophila serrata, Drosophila subauraria, Drosophila tani, Drosophila trapezifrons, Drosophila triauraria, Drosophila truncata, Drosophila vulcana, Drosophila watanabei, Drosophila fuyamai, Drosophila biarmipes, Drosophila mimetica, Drosophila pulchrella, Drosophila suzukii, Drosophila unipectinata, Drosophila lutescens, Drosophila paralutea, Drosophila prostipennis, Drosophila takahashii, Drosophila trilutea, Drosophila bifasciata, Drosophila imaii, Drosophila pseudoobscura, Drosophila saltans, Drosophila sturtevanti, Drosophila nebulosa, Drosophila paulistorum, and Drosophila willistoni. In one embodiment, the fly is Drosophila melanogaster.

As used herein, the terms “a carboxy terminal fragment of human APP”, “carboxy terminal APP fragment”, “C-terminal APP fragment”, and “CTFAPP” all refer to a fragment of human APP consisting essentially of the fragment resulting from beta secretase cleavage of an isoform of human APP resulting in C99 or C100. In some embodiments, CTFAPP contains the intracellular domain, the transmembrane domain, and a portion of the extracellular domain of APP extending out approximately to the β-secretase cleavage site. Preferably, the CTFAPP of the invention is either C99 (SEQ ID NO:2, encoded by the nucleotide sequence in SEQ ID NO:1, whereby it is noted that, because of the degeneracy of the genetic code, different nucleotide sequences can encode the same polypeptide sequence) or CTFAPP (SEQ ID NO:4, encoded by the nucleotide sequence in SEQ ID NO:3), corresponding to the C-terminal fragment of APP extending either 99 or 100 amino acids, respectively, from the C-terminus toward the N-terminus. The CTFAPP of the invention either be wild type or can possess a mutation, for example a familial mutation known or suspected to cause early onset of Alzheimer's disease or another manifestation such as cardiovascular complications. Such mutations include, but are not limited to E665D, K/M670N/L, A673T, H677R, D678N, A692G, E693G, E693Q, E693K, D694N, A713T, A713V, T714I, T714A, V715M, V715A, I716V, I716T, V717F, V717G, V717L, and L723P. Double transgenic flies also possessing a transgene encoding tau can include CTFAPP which is either wild type or bears the London mutation (V717I). Transgenic flies which do not possess the tau transgene include only mutants of CTFAPP but exclude wild type CTFAPP and the London mutation.

As used herein, the term “amyloid plaque depositions” refers to insoluble protein aggregates that are formed extracellularly by the accumulation of amyloid peptides, such as Aβ42.

As used herein, the term “signal peptide” refers to a short amino acid sequence, typically less than 20 amino acids in length, that directs proteins to or through the endoplasmic reticulum secretory pathway of a fly. “Signal peptides” used in the invention include, but are not limited to, the signal peptide of human APP695 (SEQ ID NO:5), the Drosophila signal peptides of Dint protein synonymous to “wingless (wg) signal peptide” (SEQ ID NO:6), the “argos (aos) signal peptide” (SEQ ID NO:7), the Drosophila APPL signal peptide (SEQ ID NO:8), presenilin signal peptide (SEQ ID NO:9), and windbeutel signal peptide (SEQ ID NO:10). Any signal sequence that directs proteins through the endoplasmic reticulum and results in expression of CTFAPP in a membrane where it is susceptible to gamma secretase cleavage, including variants of the above mentioned signal peptides, can be used in the present invention.

As used herein, an “amino acid linker” refers to a short amino acid sequence from about 2 to about 10 amino acids in length that is flanked by two individual peptides.

As used herein, the term “tau protein” refers to the microtubule-associated protein tau that is involved in microtubule assembly and stabilization. In neuronal tissues of Alzheimer's disease patients, tau is found in intracellular depositions of neurofibrillary tangles. Many human tau gene sequences exist. In adult human brain, six tau isoforms are produced from a single gene by alternative mRNA splicing (Goedert et al., Neuron. (1989) 3:519-26). It is noted that, because of the degeneracy of the genetic code, different nucleotide sequences can encode the same polypeptide sequence. The human gene that encodes the human tau protein contains 11 exons as described by Andreadis, A. et al., Biochemistry, 31 (43):10626-10633 (1992), herein incorporated by reference. In adult human brain, six tau isoforms are produced from a single gene by alternative mRNA splicing. They differ from each other by the presence or absence of 29- or 58-amino-acid inserts located in the amino-terminal half and a 31-amino acid repeat located in the carboxyl-terminal half. Inclusion of the latter, which is encoded by exon 10 of the tau gene, gives rise to the three tau isoforms which each have 4 repeats. As used herein, the term “tau protein” includes various tau isoforms produced by alternative mRNA splicing as well as mutant forms of human tau proteins as described in SEQ ID NO:11, SEQ ID NO:12, SEQ ID NO:13, and SEQ ID NO:14. In one embodiment, the tau protein used to generate the double transgenic fly is represented by SEQ ID NO:15 (amino acid sequence) and SEQ ID NO:16 (nucleotide sequence). This isoform contains Tau exons 2 and 3 as well as four microtuble-binding repeats. In the normal human cerebral cortex, there is a slight preponderance of 3 repeat over 4 repeat tau isoforms. These repeats and some adjoining sequences constitute the microtubule-binding domain of tau (Goedert, et al., 1998 Neuron 21, 955-958). In neuronal tissues of Alzheimer's disease patients, tau is hyperphosphorylated and adopts abnormal and/or pathological conformations detectable using conformation-dependent antibodies, such as MCI and ALZ50 (Jicha G. A., et al., Journal of Neuroscience Research 48:128-132 (1997)). Thus, “tau protein”, as used herein, includes tau protein recognized by these conformation-specific antibodies.

The invention further contemplates, as equivalents of these tau sequences, mutant sequences that retain the biological effect of tau of forming neurofibrillary tangles. Therefore, “tau protein”, as used herein, also includes tau proteins containing mutations and variants. These mutations include but are not limited to: Exon 10+12 “Kumamoto pedigree” (Yasuda et al., (2000) Ann Neurol. 47:422-9); I260V (Grover et al., Exp Neurol. 2003 November; 184:131-40); G272V (Hutton et al., 1998 Nature 393:702-5; Heutink et al., (1997) Ann Neurol. 41:150-9; Spillantini et al., (1996) Acta Neuropathol (Berl). 1996 July; 92:42-8); N279K (Clark et al., (1998) Proc Natl Acad Sci USA 95:13103-13107; D'Souza et al., (1999) Proc Natl Acad Sci USA 96:5598-5603; Reed et al., (1997) Ann Neurol. 1997 42:564-72; Hasegawa et al., (1999) FEBS Letters 443:93-96; Hong et al., (1998) Science 282: 1914-17); delK280 (Rizzu et al., (1999) Am Hum Genet. 64:414-421; D'Souza et al., (1999) Proc Natl Acad Sci USA 96:5598-5603); L284L (D'Souza et al., (1999) Proc Natl Acad Sci USA 96:5598-5603); P301L (Hutton et al., 1998 Nature 393:702-5; Heutink et al., (1997) Ann Neurol. 41:150-9; Spillantini et al., (1996) Acta Neuropathol (Berl) (1996) 92:42-8; Hasegawa et al., (1998) FEBS Lett. 1998 437(3):207-10; Nacharaju et al., (1999) FEBS Letters 447:195-199); P301S (Bugiani (1999) J Neuropathol Exp Neurol 58:667-77; Goedert et al., (1999) FEBS Letters 450: 306-311); S305N (Iijima et al., (1999) Neuroreport 10:497-501; Hasegawa et al., (1998) FEBS Lett. 1998 437:207-10; D'Souza et al., (1999) Proc Natl Acad Sci USA 96:5598-5603); S305S (Stanford et al., Brain, 123, 880-893, 2000); S305S (Wszolek et al., Brain. 2001 124:1666-70); V337M (Poorkaj et al., (1998) Ann Neurol. 1998 43:815-25; Spillantini et al., (1998) American Journal of Pathology 153: 1359-1363; Sumi et al., (1992) Neurology. 42:120-7; Hasegawa et al., (1998) FEBS Lett. 1998 437:207-10); G389R (Murrell et al., J Neuropathol Exp Neurol. (1999) 58:1207-26; Pickering-Brown, et al., Ann Neurol. (2000) 48:859-67); R406W (Hutton et al., (1998) Nature 393:702-5; Reed et al., (1997) Ann Neurol. 42:564-72; Hasegawa et al., (1998) FEBS Lett. 437:207-10); 3′Ex10+3, GtoA (Spillantini et al., (1998) American Journal of Pathology 153:1359-1363; Spillantini et al., (1997) Proc Natl Acad Sci USA. 94:4113-8); 3′Ex10+16 (Baker et al., (1997) Annals of Neurology 42:794-798; Goedert et al., (1999b) Nature Medicine 5:454-457; Hutton et al., (1998) Nature 393:702-705); 3′Ex10+14 (Hutton et al., (1998) Nature 393:702-705; Lynch et al., (1994) Neurology 44:1878-1884); 3′Ex10+13 (Hutton et al., (1998) Nature 393:702-705).

The invention furthermore includes the use of tau genes containing sequence polymorphisms (see, for example, Table 1).

TABLE 1
Polymorphisms identified within the human tau gene.
Underlined polymorphisms are inherited as a part of extended haplotype 2.
In case of exons skipped in the brain mRNA (exon 4a, 6, 8) locations
of polymorphic sites are counted from the first nucleotide of the exon.

| Exon/Intron | Polymorphisms |
|---|---|
| E1 | 5′ UTR-13 a--> g |
| I1 | nt − 93 t --> c |
| I2 | nt + 18 c --> t |
| I3 | nt + 9 a --> g |
| I3 | nt − 103 t --> a (very rare on H1) |
| I3 | nt − 94a -->t (very rare on H1) |
| E4a | n + 232 C --> T (CCG/CTG; P/L) |
| E4a | n + 480 G --> A (GAC/AAC; R/N) |
| E4a | n + 482 C --> T (GAC/GAT; N/N) |
| E4a | n + 493 T --> C (GTA/GCA; V/A) |
| E4a | n316 A --> G (CAA/CGA, Q/Q) |
| I4a | nt − 72 t --> c |
| E6 | n + 139 C --> T (CAC/TAC H/Y) (very common) |
| E6 | n + 157 T --> C (ACT/ACC S/P) |
| I6 | nt + 67 a --> g |
| I6 | nt + 105 t --> c |
| E7 | P176P (G --> A) |
| E8 | n + 5 T --> C (ACT/ACC, T/T) |
| I8 | nt − 26 g --> a |
| E9 | A227A (GCA/GCG) |
| E9 | N255N (AAT/AAC) |
| E9 | P270P (CCG/CCA) |
| I9 | nt − 47 c --> a (very rare on H1) |
| I9 | Δ238 bp |
| I11 | nt + 34 g --> a |
| I11 | nt + 90 g --> a |
| I11 | nt + 296 c --> t |
| I13 | nt + 34 t --> c |

The invention also contemplates the use of tau proteins or genes from other animals, including but not limited to mice (Lee et al., Science 239:285-8 (1988)), rats (Goedert et al., Proc. Natl. Acad. Sci. U.S.A. 89:1983-1987 (1992)), Bos taurus (Himnimler et al., Mol. Cell. Biol. 9:1381-1388 (1989)), Drosophila melanogaster (Heidary & Fortini, Mech. Dev. 108:171-178 (2001)) and Xenopus laevis (Olesen et al., Gene 283:299-309 (2002)). The tau genes from other animals may additionally contain mutations equivalent to those previously described. Equivalent positions can be identified by sequence alignment, and equivalent mutations can be introduced by means of site-directed mutagenesis or other means known in the art.

As used herein, the term “neurofibrillary tangles” refers to insoluble twisted fibers that form intracellularly and that are composed mainly of tau protein.

As used herein, the term “operatively linked” refers to a juxtaposition wherein the components described are in a relationship permitting them to function in their intended manner. An expression control sequence “operatively linked” to a coding sequence is ligated in such a way that expression of the coding sequence is achieved under conditions compatible with the activity of the control sequence.

As used herein, the term “expression control sequence” refers to promoters, enhancer elements, and other nucleic acid sequences that contribute to the regulated expression of a given nucleic acid sequence. The term “promoter” refers to DNA sequences recognized by RNA polymerase during initiation of transcription and can include enhancer elements. As used herein, the term “enhancer element” refers to a cis-acting nucleic acid element, which controls transcription initiation from homologous as well as heterologous promoters independent of distance and orientation. Preferably, an “enhancer element” also controls the tissue and temporal specification of transcription initiation. In particular embodiments, enhancer elements include, but are not limited to, the UAS control element. “UAS” as used herein, refers to an Upstream Activating Sequence recognized and bound by the GAL4 transcriptional activator. The term “UAS control element”, as used herein, refers to a UAS element that is activated by GAL4 transcriptional regulator protein. Expression control sequences of the invention preferably are selected for their ability to drive expression in a tissue-specific manner. An expression control sequence alternatively can be derived from ubiquitously expressed genes like tubulin, actin, or ubiquitin. In yet other embodiments, the expression control sequence comprises a tetracycline-controlled transcriptional activator (tTA) responsive regulatory element. Optionally, the transgenic flies comprising the tau and CTFAPP transgenes further comprise a tTA gene.

A “tissue-specific” expression control sequence, as used herein, refers to expression control sequences that drive expression in one tissue or a subset of tissues, while being essentially inactive in at least one other tissue. “Essentially inactive” means that the expression of a sequence operatively linked to a tissue-specific expression control sequence is less than 5% of the level of expression of that sequence in that tissue where the expression control sequence is active. Preferably, the level of expression in the tissue is less than 1% of the maximal activity, or there is no detectable expression of the sequence in the tissue. “Tissue-specific expression control sequences” include those that are specific for organs such as the eye, wing, notum, brain, as well as tissues of the central and peripheral nervous systems. Tissue-specific expression control sequences of the invention either can be used as a “driver” in the GAL4-UAS system, or alternatively can be inserted upstream from a transgene to control its expression in a cis acting manner.

Examples of tissue specific control sequences include but are not limited to: promoters/enhancers important in eye development, such as sevenless (Bowtell et al., Genes Dev. 2:620-34 (1988)), eyeless (Bowtell et al., Proc. Natl. Acad. Sci. U.S.A. 88:6853-7 (1991)), and GMR/glass (Quiring et al., Science 265:785-9 (1994)); promoters/enhancers derived from any of the rhodopsin genes, that are useful for expression in the eye; enhancers/promoters derived from the dpp, vestigial, or wingless genes useful for expression in the wing (Staehling-Hampton et al., Cell Growth Differ. 5:585-93 (1994); Kim et al., Nature 382:133-8 (1996); Giraldez et al., Dev. Cell 2:667-676 (2002)); promoters/enhancers specific for nerve, e.g., elav (Yao and White, J. Neurochem. 63:41-51 (1994)) which is specific for pan-neuronal expression in post-mitotic neurons, scabrous (sca) (Song et al., Genetics 162:1703-24 (2002) which is specific for pan-neuronal expression in neuroblasts to neurons, APPL (Martin-Morris and White, Development 110: 185-95 (1990)), Nervana 2 (Nrv2)(Sun et al., Proc. Nat'l. Acad. Sci. U.S.A. 96:10438-43 (1999)) which is specific for expression in the central nervous system, Cha (Barber et al., J. Comp. Neurol. 22:533-43 (1989)) which is specific for cholinergic neurons, TH (Friggi-Grelin et al., J. Neurobiol. 54:618-27 (2003)) which is specific for dopaminergic neurons, CaMKII (Takmatsu et al., Cell Tissue Res. 310:237-52 (2002)) which is specific for central nervous system of embryos and larvae as well as brain, throacic ganglion and gut of adult, P (Gendre et al., Development 131:83-92 (2004)) which is specific for pharangeal sensory neurons, Dmej2 (Mao et al., Proc. Natl. Acad. Sci. USA 101:198-203 (2004), GAL4 line named “P247”) and OK107 (Lee et al., Development 126:4065-4076 (1999)) which are specific for mushroom bodies of the brain, C164 (Torroja et al., J. Neurosci. 19:7793-7803 (1999) which is specific for motor neurons, and promoters/enhancers derived from other neural-specific genes; and gcm (Dumstrei et al., J. Neurosci. 23:3325-35 (2003)) which is specific for glial cells; all of which references are incorporated by reference herein. Other examples of expression control sequences include, but are not limited to the heat shock promoters/enhancers from the hsp70 and hsp83 genes, useful for temperature induced expression; and promoters/enhancers derived from ubiquitously expressed genes, such as tubulin, actin, or ubiquitin.

As used herein, the term “phenotype” with respect to a transgenic fly refers to an observable and/or measurable physical, behavioral, or biochemical characteristic of a fly. The term “altered phenotype” or “change in phenotype” as used herein, refers to a phenotype that has changed measurably or observably relative to the phenotype of a wild-type fly. Examples of altered phenotypes include behavioral phenotypes, such as appetite, mating behavior, and/or life span; morphological phenotypes, such as rough eye phenotype, concave wing phenotype, or any different shape, size, color, growth rate or location of an organ or appendage, or different distribution, and/or characteristic of a tissue or cell, as compared to the similar characteristic observed in a control fly; and locomotor dysfunction phenotypes, such as reduced climbing ability, reduced walking ability, reduced flying ability, decreased speed or acceleration, abnormal trajectory, abnormal turning, and abnormal grooming. An altered phenotype is a phenotype that has changed by a measurable amount, e.g., by at least a statistically significant amount, preferably by at least 1%, 5%, 10%, 20%, 30%, 40%, or 50% relative to the phenotype of a control fly. As used herein, “a synergistic altered phenotype” or “synergistic phenotype,” refers to a phenotype wherein a measurable and/or observable physical, behavioral, or biochemical characteristic of a fly is more than the sum of its components.

The term “phenotype” with respect to a cell obtained from a transgenic fly of the invention, for example a cell in a primary culture obtained from such a fly, refers to an observable and/or measurable physical, physiological, or biochemical characteristic of the cell. A cell phenotype can be, for example, any morphological property of the cell, such as size, shape, aggregation state, or any ultrastructural property of the interior of the cell, such as distribution or appearance of an organelle or molecular assemblage, organization of the cytoskeleton, presence or appearance of intracellular microfibrillary tangles, or presence or appearance of extracellular plaques. Cell phenotype can also include any aspect of cell motility, attachment to substratum or other cells, extension of structures such as axons or dendrites, axonal transport, exocytosis, endocytosis, secretion, neurotransmitter release, macromolecular synthesis or breakdown, metabolism, sensitivity to oxidative stress, levels of biochemical substrates or products, levels of phosphorylation of proteins (e.g., altered phosphorylation of tau or β-amyloid induced altered phosphorylation of tau), transport activity, electrophysiological properties, DNA synthesis, gene transcription, protein synthesis, cell cycle phenomena, viability, and the like.

As used herein, the “rough eye” phenotype is characterized by loss of rhabdomeres, irregular ommatidial packing, occasional ommatidial fusions, and missing bristles that can be caused by degeneration of neuronal cells. The eye can become rough in texture relative to its appearance in wild type flies, and can be easily observed by microscope. Neurodegeneration is readily observed and quantified in a fly's compound eye, which can be scored without any preparation of the specimens (Femandez-Funez et al., 2000, Nature 408:101-106; Steffan et. al, 2001, Nature 413:739-743; Agrawal et al., 2005, Proc. Natl. Acad. Sci. USA 102:3777-3781). This organism's eye is composed of a regular trapezoidal arrangement of seven visible rhabdomeres produced by the photoreceptor neurons of each Drosophila ommatidium. Expression of mutant transgenes specifically in the Drosophila eye leads to a progressive loss of rhabdomeres and subsequently a rough-textured eye, which can be expressed quantitatively, for example, as the number of rhabdomeres per ommatidium (Fernandez-Funez et al., 2000; Steffan et. al, 2001). Administration of therapeutic compounds to these organisms slows the photoreceptor degeneration and improves the rough-eye phenotype (Steffan et. al, 2001).

As used herein, the “concave wing” phenotype is characterized by abnormal folding of the fly wing such that wings are bent upwards along their long margins.

As used herein, “locomotor dysfunction” refers to a phenotype where flies have a deficit in motor activity, movement, or response to a stimulus (e.g., at least a statistically significant difference, or at least a 10% difference in a measurable parameter) as compared to control flies. Motor activities include flying, climbing, crawling, and turning. In addition, movement traits where a deficit can be measured include, but are not limited to: i) average total distance traveled over a defined period of time; ii) average distance traveled in one direction over a defined period of time; iii) average speed (average total distance moved per time unit); iv) distance moved in one direction per time unit; v) acceleration (the rate of change of velocity with respect to time; vi) turning; vii) stumbling; viii) spatial position of a fly to a particular defined area or point; ix) path shape of the moving fly; and x) undulations during larval movement; xi) rearing or raising of larval head; and xii) larval tail flick. Examples of movement traits characterized by spatial position include, without limitation: (1) average time spent within a zone of interest (e.g., time spent in bottom, center, or top of a container; number of visits to a defined zone within container); and (2) average distance between a fly and a point of interest (e.g., the center of a zone). Examples of path shape traits include the following: (1) angular velocity (average speed of change in direction of movement); (2) turning (angle between the movement vectors of two consecutive sample intervals); (3) frequency of turning (average amount of turning per unit of time); and (4) stumbling or meander (change in direction of movement relative to the distance). Turning parameters can include smooth movements in turning (as defined by small degrees rotated) and/or rough movements in turning (as defined by large degrees rotated). Locomotor phenotypes can be analyzed using methods described, for example, in U.S. Application Nos. 2004/0076583, 2004/0076318, and 2004/0076999, each of which is hereby incorporated by reference in its entirety.

A phenoprofile of a test or reference population is determined by measuring traits of the population. The present invention allows simultaneous measurement of multiple traits of a population. Although a single trait may be measured, multiple traits can also be measured. For example, at least 2, at least 3, at least 4, at least 5, at least 7 or at least 10 traits can be assessed for a population. The traits measured can be solely movement traits, solely behavioral traits solely morphological traits or a mixture of traits in multiple categories. In some embodiments at least one movement trait and at least one non-movement trait are assessed.

As used herein, a “control fly” refers to a larval or adult fly of the same genotype of the transgenic fly as to which it is compared, except that the control fly either i) does not comprise one or both of the transgenes present in the transgenic fly, or ii) has not been administered a candidate agent, or iii) does not have a driver for the GAL4-UAS system.

As used herein, the term “candidate agent” refers to a biological or chemical compound that when administered to a transgenic fly has the potential to modify the phenotype of the fly, e.g. partial or complete reversion of the altered phenotype towards the phenotype of a wild type fly. “Agents” as used herein can include any recombinant, modified or natural nucleic acid molecule, library of recombinant, modified or natural nucleic acid molecules, synthetic, modified or natural peptide, library of synthetic, modified or natural peptides; and any organic or inorganic compound, including small molecules, or library of organic or inorganic compounds, including small molecules.

As used herein, the term “small molecule” refers to compounds having a molecular mass of less than 3000 Daltons, preferably less than 2000 or 1500, more preferably less than 1000, and most preferably less than 600 Daltons. Preferably but not necessarily, a small molecule is a compound other than an oligopeptide.

As used herein, a “therapeutic agent” refers to an agent that ameliorates one or more of the symptoms of a neurodegenerative disorder such as Alzheimer's disease in mammals, particularly humans. A therapeutic agent can reduce one or more symptoms of the disorder, delay onset of one or more symptoms, or prevent or cure the disease.

### DESCRIPTION

I. Generation of Transgenic Drosophila

A transgenic fly that carries a transgene encoding CTFAPP, as well as a double transgenic fly carrying transgenes encoding both the tau protein and CTFAPP, are disclosed. The transgenic flies provide a model for neurodegenerative disorders such as Alzheimer's disease, which is characterized by an extracellular accumulation of Aβ42 peptide and an intracellular deposition of a hyperphosphorylated form of microtubule-associated protein tau. The transgenic flies of the present invention can be used to screen for therapeutic agents effective in the treatment of Alzheimer's disease.

Flies such as Drosophila possess γ-secretase activity but do not possess β-secretase activity. Therefore, expression of human APP alone in transgenic flies would not result in the formation of Aβ42 or Aβ40 peptides. However, since the N-terminus of CTFAPP approximates the N-terminus resulting from beta secretase cleavage, expression of CTFAPP alone in a transgenic fly will result in the formation of Aβ42, Aβ40, and similar peptides due to the action of γ-secretase in the fly upon CTFAPP as substrate. Therefore, flies of the present invention are a suitable model for studying the processing, trafficking, and accumulation of Aβ42 and resulting neurodegeneration as well as the effects of mutations and variations in the CTFAPP encoding region of the APP gene or of mutations and variations in the tau gene.

The transgenic flies of the present invention can be generated by any means known to those skilled in the art. Methods for production and analysis of transgenic Drosophila strains are well established and described in Brand et al., Methods in Cell Biology 44:635-654 (1994); Hay et al., Proc. Natl. Acad. Sci. USA 94(10):5195-200 (1997); and in Drosophila: A Practical Approach (ed. D. B. Roberts), pp 175-197, IRL Press, Oxford, UK (1986), herein incorporated by reference in their entireties.

In general, to generate a transgenic fly, a transgene of interest is stably incorporated into a fly genome. Any fly can be used, however a preferred fly of the present invention is a member of the Drosophilidae family. An exemplary fly is Drosophila melanogaster.

A variety of transformation vectors are useful for the generation of the transgenic flies of the present invention, and include, but are not limited to, vectors that contain transposon sequences, which mediate random integration of a transgene into the genome, as well as vectors that use homologous recombination (Rong and Golic, Science 288:2013-2018 (2000)). A preferred vector of the present invention is pUAST (Brand and Perrimon, Development 118:401-415 (1993)) which contains sequences from the transposable P-element which mediate insertion of a transgene of interest into the fly genome. Another preferred vector is PdL, which is able to yield doxycycline-dependent overexpression (Nandis, Bhole and Tower, Genome Biology 4 (R8):1-14, (2003)). Yet another preferred vector is pExP-UAS because of its ease of cloning and mapping genomic location. Two particular vectors used in the instant invention are pExP-UAS:CTF-I (SEQ ID NO:17) and pExP-UAS:CTF-II (SEQ ID NO:18). pExP-UAS:CTF-I encodes the signal sequence of human APP, CTFAPP, and a myc tag. pExP-UAS:CTF-II encodes the signal sequence of a Drosophila cuticle protein (Vinc), CTFAPP, and a 3×HA tag.

P-element transposon mediated transformation is a commonly used technology for the generation of transgenic flies and is described in detail in Spradling, P-element mediated transformation, in Drosophila: A Practical Approach (ed. D. B. Roberts), pp 75-197, IRL Press, Oxford, UK (1986), herein incorporated by reference. Other transformation vectors based on transposable elements include, for example, the hobo element (Blackman et al., Embo J. 8:211-7 (1989)), the mariner element (Lidholm et al., Genetics 134:859-68 (1993)), the hermes element (O'Brochta et al., Genetics 142:907-14 (1996)), the Minos element (Loukeris et al., Proc. Natl. Acad. Sci. USA 92:9485-9 (1995)), or the PiggyBac element (Handler et al., Proc. Natl. Acad. Sci. USA 95:7520-5 (1998)). In general, the terminal repeat sequences of the transposon that are required for transposition are incorporated into a transformation vector and arranged such that the terminal repeat sequences flank the transgene of interest. It is preferred that the transformation vector contains a marker gene used to identify transgenic animals. Commonly used, marker genes affect the eye color of Drosophila, such as derivatives of the Drosophila white gene (Pirrotta V., & C. Brockl, EMBO J. 3(3):563-8 (1984)) or the Drosophila rosy gene (Doyle W. et al., Eur. J. Biochem. 239(3):782-95 (1996)) genes. Any gene that results in a reliable and easily measured phenotypic change in transgenic animals can be used as a marker. Examples of other marker genes used for transformation include the yellow gene (Wittkopp P. et al., Curr Biol. 12(18):1547-56 (2002)) that alters bristle and cuticle pigmentation; theforked gene (McLachlan A., Mol Cell Biol. 6(1):1-6 (1986)) that alters bristle morphology; the Adh+ gene used as a selectable marker for the transformation of Adh− strains (McNabb S. et al., Genetics 143(2):897-911 (1996)); the Ddc+ gene used to transform Ddc^{ts2} mutant strains (Scholnick S. et al., Cell 34(1):37-45 (1983)); the lacZ gene of E. coli; the neomycin^{R} gene from the E. coli transposon Tn5; and the green fluorescent protein (GFP; Handler and Harrell, Insect Molecular Biology 8:449-457 (1999)), which can be under the control of different promoter/enhancer elements, e.g. eyes, antenna, wing and leg specific promoter/enhancers, or the poly-ubiquitin promoter/enhancer elements.

Plasmid constructs for introduction of the desired transgene are coinjected into Drosophila embryos having an appropriate genetic background, along with a helper plasmid that expresses the specific transposase needed to incorporate the transgene into the genomic DNA. Animals arising from the injected embryos (G0 adults) are selected, or screened manually, for transgenic mosaic animals based on expression of the marker gene phenotype and are subsequently crossed to generate fully transgenic animals (G1 and subsequent generations) that will stably carry one or more copies of the transgene of interest.

Binary systems are commonly used for the generation of transgenic flies, such as the UAS/GAL4 system. This is a well established system which employs the UAS upstream regulatory sequence for control of promoters by the yeast GAL4 transcriptional activator protein, as described in Brand and Perrimon, Development 118:401-15 (1993)) and Rorth et al, Development 125:1049-1057 (1998), herein incorporated by reference in their entireties. In this approach, transgenic Drosophila, termed “target” lines, are generated where the gene of interest (e.g., the transgene encoding C-terminal APP fragment or tau)) is operatively linked to an appropriate promoter (e.g., hsp70 TATA box, see Brand and Perrimon, Development 118:401-15 (1993)) controlled by UAS. Other transgenic Drosophila strains, termed “driver” lines, are generated where the GAL4 coding region is operatively linked to promoters/enhancers that direct the expression of the GAL4 activator protein in specific tissues, such as the eye, antenna, wing, or nervous system. The gene of interest is not expressed in the “target” lines for lack of a transcriptional activator to “drive” transcription from the promoter joined to the gene of interest. However, when the UAS-target line is crossed with a GAL4 driver line, the gene of interest is induced. The resultant progeny display a specific pattern of expression that is characteristic for the GAL4 line.

The technical simplicity of this approach makes it possible to sample the effects of directed expression of the gene of interest in a wide variety of tissues by generating one transgenic target line with the gene of interest, and crossing that target line with a panel of pre-existing driver lines. Numerous GAL4 driver Drosophila strains with specific drivers have been described in the literature and others can readily be prepared using established techniques (Brand and Perrimon, Development 118:401-15 (1993)). Driver strains for use with the invention include, for example, apterous-GAL4 for expression in wings, brain, and interneurons; elav-GAL4 for pan-neuronal expression in post-mitotic neurons; scabrous-GAL4 for pan-neuronal expression in the developing nervous system from neuroblasts to neurons; sevenless-GAL4, eyeless-GAL4, and GMR-GAL4 for expression in eyes; Nervana 2-GAL4 for expression in the central nervous system; Cha—(choline acetyltransferase) GAL4 for expression in cholinergic neurons, TH—(tyrosine hydroxylase) for expression in dopaminergic neurons; CaMKII-(calmodulin dependent kinase II) for expression in the central nervous system of embryos and larvae as well as the brain, throacic ganglion, and gut of adults; P-GAL4 for expression in pharangeal sensory neurons; and gcm-GAL4 for expression in glial cells.

The present invention discloses transgenic flies that have incorporated into their genome a DNA sequence that encodes CTFAPP, optionally fused to a DNA sequence for a signal peptide. Some embodiments are double transgenic flies which comprise a DNA sequence that encodes the tau protein as well as a DNA sequence encoding CTFAPP.

Generation of transgenic flies containing single transgenes can be performed using any standard means known to those skilled in the art. To generate the double transgenic fly, transgenic flies that express either CTFAPP or the tau protein are independently made and then crossed to generate a fly that expresses both proteins.

One or more transgenes of a transgenic fly can be driven either directly by a selected promoter or by means of the UAS/GAL4 system. In a preferred embodiment, transgenic Drosophila are produced using the UAS/GAL4 control system. Briefly, to generate a transgenic fly that expresses a transgene (e.g., CTFAPP), a DNA sequence encoding the transgene is cloned into a vector such that the sequence is operatively linked to the GAL4 responsive element UAS. Vectors containing UAS elements are commercially available, such as the pUAST vector (Brand and Perrimon, Development 118:401-415 (1993)), which places the UAS sequence element upstream of the transcribed region. The DNA is cloned using standard methods (Sambrook et al., Molecular Biology: A laboratory Approach, Cold Spring Harbor, N.Y. (1989); Ausubel, et al., Current protocols in Molecular Biology, Greene Publishing, Y, (1995)) and is described in more detail under the Molecular Techniques section of the present application. After cloning the DNA into appropriate vector, such as pUAST, the vector is injected into Drosophila embryos (e.g. yw embryos) by standard procedures (Brand et al., Methods in Cell Biology 44:635-654 (1994); Hay et al., Proc. Natl. Acad. Sci. USA 94:5195-200 (1997)) to generate transgenic Drosophila.

When the binary UAS/GAL4 system is used, the transgenic progeny can be crossed with Drosophila driver strains to assess the presence of an altered phenotype. A preferred Drosophila comprises the eye specific driver strain GMR-GAL4, which enables identification and classification of transgenics flies based on the severity of the rough eye phenotype. For example, expression of tau in Drosophila eye results in the rough eye phenotype, which can be easily observed by microscope. The severity of the rough eye phenotype exhibited by a transgenic line, can be classified as strong, medium, or weak. The weak or mild lines have a rough, disorganized appearance covering the ventral portion of the eye. The medium severity lines show greater roughness over the entire eye, while in strong severity lines the entire eye seems to have lost/fused many of the ommatidia and interommatidial bristles, and the entire eye has a smooth, glossy appearance; additionally, necrotic patches can be detected throughout the eye.

To generate a transgenic fly that expresses CTFAPP, a DNA sequence encoding CTFAPP is ligated in frame to a DNA sequence encoding a signal peptide such that CTFAPP can be inserted into a cell membrane. The signal sequence is directly linked to the CTFAPP coding sequence or indirectly linked by using a DNA linker sequence, for example of 3, 6, 9, 12, or 15 nucleotides. A signal peptide that directs proteins to or through the endoplasmic reticulum secretory pathway of Drosophila is used. Preferred signal peptides of the present invention are signal peptides from human APP (SEQ ID NO:5), wingless (wg) (SEQ ID NO:6), argos (aos) (SEQ ID NO:7), Drosophila APPL (SEQ ID NO:8), presenilin (psn) (SEQ ID NO:9), and windbeutel (SEQ ID NO:10) and Vinc.

The DNA encoding CTFAPP is linked to a signal sequence by standard ligation techniques and is then cloned into a vector such that the sequence is operatively linked to the GAL4 responsive element UAS. A preferred transformation vector for the generation of CTFAPP transgenic flies is the pUAST vector (Brand and Perrimon, Development 118:401-415 (1993)). As described for the generation of tau transgenic flies, the vector is injected into Drosophila embryos (e.g. yw embryos) by standard procedures (Brand et al., Meth. in Cell Biol. 44:635-654 (1994)); Hay et al., Proc. Natl. Acad. Sci. USA 94:5195-200 (1997)) and progeny are then selected and crossed based on the phenotype of the selected marker gene. When the binary UAS/GAL4 system is used, the transgenic progeny can be crossed with Drosophila driver strains to assess the presence of an altered phenotype. Preferred Drosophila driver strains are GMR-GAL4 (eye) and elav-GAL4 (CNS).

To assess an eye phenotype (e.g., rough eye phenotype) a GMR-GAL4 driver strain can be used in the cross. Ectopic overexpression of CTFAPP in Drosophila eye is believed to disrupt the regular trapezoidal arrangement of the photoreceptor cells of the ommatidia (identical single units, forming the Drosophila compound eye), the severity of which is believed to depend on transgene copy number and expression levels. To evaluate locomotor and behavioral phenotypes (e.g., climbing assay), an elav-GAL4 driver strain is used in the cross. Ectopic overexpression of CTFAPP in Drosophila central nervous system (CNS) is believed to result in locomotor deficiencies, such as impaired movement, climbing and flying.

Once single transgenic flies are produced, the flies can be crossed with each other by mating. Flies are crossed according to conventional methods. When the binary UAS/GAL4 system is used, the fly is crossed with an appropriate driver strain and the altered phenotype assessed, as described above, transgenic flies are classified by assessing phenotypic severity. For example, as disclosed herein, the combination of tau and CTFAPP transgenes is believed to produce a synergistic effect on the eye.

Several factors can be varied to alter the specificity and intensity of transgene expression. Sequence variants of the transgene or combination of transgenes can be used to alter phenotype. Different expression drivers, e.g., tissue specific promoters used either alone or in conjunction with the GAL4 system, can be used to affect either the tissue specificity or intensity of expression. The temperature of development can also be varied, which can affect either tissue distribution or intensity of expression. In general, higher temperatures drive stronger expression of the transgene.

Expression of tau and CTFAPP proteins in transgenic flies is confirmed by standard techniques, such as Western blot analysis or by immunostaining of fly tissue cross-sections, both of which are described below.

Western blot analysis is performed by standard methods. Briefly, as means of example, to detect expression of CTFAPP or tau by Western blot analysis, whole flies, or fly heads (e.g., 80-90 heads) are collected and placed in an eppendorf tube on dry ice containing 100 μl of 2% SDS, 30% sucrose, 0.718 M Bis-Tris, 0.318 M Bicine, with “Complete” protease inhibitors (Boehringer Mannheim), then ground using a mechanical homogenizer. Samples are heated for 5 min at 95° C., centrifuged for 5 min at 12,000 rpm, and supernatants are transferred into a fresh eppendorf tube. 5% β-mercaptoethanol and 0.01% bromphenol blue are added and samples are boiled prior to loading on a separating gel. Approximately 200 ng of total protein extract is loaded for each sample on a 15% Tricine/Tris SDS PAGE gel containing 8M Urea. After separating, samples are then transferred to PVDF membranes (BIO-RAD, 162-0174) and the membranes are subsequently boiled in PBS for 3 min. Anti-tau antibody (e.g. T14 (Zymed) and AT100 (Pierce-Endogen), anti-APP antibody (e.g. 6E10 (Senetek PLC Napa, Calif.), or anti-Aβ42 is hybridized, generally at a concentration of 1:2000, in 5% non-fat milk, 1×PBS containing 0.1% Tween 20, for 90 min at room temperature. Samples are washed 3 times for 5 min., 15 min., and 15 min. each, in 1×PBS-0.1% Tween-20. Labeled secondary antibody, (for example, anti-mouse-HRP from Amersham Pharmacia Biotech, NA 931) is prepared, typically at a concentration of 1:2000, in 5% non-fat milk, 1×PBS containing 0.1% Tween 20, for 90 min at room temperature. Samples are then washed 3 times for 5 min., 15 min., and 15 min. each, in, 1×PBS-0.1% Tween-20. Protein is then detected using the appropriate method. For example, when anti-mouse-HRP is used as the conjugated secondary antibody, ECL (ECL Western Blotting Detection Reagents, Amersham Pharmacia Biotech, # RPN 2209) is used for detection.

As a manner of confirming protein expression in transgenic flies, immunostaining of Drosophila organ cross sections or whole mount is performed. Such a method is of particular use to confirm the presence of hyperphosphorylated tau, which is a modified form of the tau protein that is present in non-diseased tissue. Hyperphosphorylated tau exhibits altered pathological conformations as compared to tau protein and is present in diseased tissue from patients with certain neurodegenerative disorders, such as Alzheimer's disease.

Cross sections of Drosophila organs can be made by any conventional cryosectioning, such as the method described in Wolff, Drosophila Protocols, CSHL Press (2000), herein incorporated by reference. Cryosections can then be immunostained for detection of tau and C-terminal APP fragment peptides using methods well known in the art. In a preferred embodiment, the Vectastain ABC Kit (which comprises biotinylated anti-mouse IgG secondary antibody, and avidin/biotin conjugated to the enzyme Horseradish peroxidase H (Vector Laboratories) is used to identify the protein. In other embodiments the secondary antibody is conjugated to a fluorophore. Briefly, cryosections are blocked using normal horse serum, according to the Vectastain ABC Kit protocol. The primary antibody, recognizing CTFAPP or tau, is typically used at a dilution of 1:3000 and incubation with the secondary antibody is done in PBS/1% BSA containing 1-2% normal horse serum, also according to the Vectastain ABC Kit protocol. The procedure for the ABC Kit is followed; incubations with the ABC reagent are done in PBS/0.1% saponin, followed by 4×10 minute washes in PBS/0.1% saponin. Sections are then incubated in 0.5 ml per slide of the Horseradish Peroxidase H substrate solution, 400 ug/ml 3,3′-diaminobenzidene (DAB), 0.006% H 202 in PBS/0.1% saponin, and the reaction is stopped after 3 min with 0.02% sodium azide in PBS. Sections are rinsed several times in PBS and dehydrated through an ethanol series before mounting in DPX (Fluka).

Exemplary antibodies that can be used to immunostain cross sections include but are not limited to, the monoclonal antibody 6E10 (Senetek PLC Napa, Calif.) that recognizes Aβ42 peptide and anti-tau antibodies ALZ50 and MCI (Jicha G A, et al., J. of Neurosci. Res. 48:128-132 (1997)).

Alternatively, antibodies for use in the present invention that recognize C-terminal APP fragment and tau can be made using standard protocols known in the art (See, for example, Antibodies: A Laboratory Manual ed. by Harlow and Lane (Cold Spring Harbor Press: 1988)). A mammal, such as a mouse, hamster, or rabbit can be immunized with an immunogenic form of the protein (e.g., a Aβ42 or tau polypeptide or an antigenic fragment which is capable of eliciting an antibody response). Immunogens for raising antibodies are prepared by mixing the polypeptides (e.g., isolated recombinant polypeptides or synthetic peptides) with adjuvants. Alternatively, C-terminal APP fragment or tau polypeptides are made as fusion proteins to larger immunogenic proteins. Polypeptides can also be covalently linked to other larger immunogenic proteins, such as keyhole limpet hemocyanin. Alternatively, plasmid or viral vectors encoding C-terminal APP fragment or tau, or a fragment of these proteins, can be used to express the polypeptides and generate an immune response in an animal as described in Costagliola et al., J. Clin. Invest. 105:803-811 (2000), which is incorporated herein by reference. In order to raise antibodies, immunogens are typically administered intradermally, subcutaneously, or intramuscularly to experimental animals such as rabbits, sheep, and mice. In addition to the antibodies discussed above, genetically engineered antibody derivatives can be made, such as single chain antibodies.

The progress of immunization can be monitored by detection of antibody titers in plasma or serum. Standard ELISA, flow cytometry or other immunoassays can also be used with the immunogen as antigen to assess the levels of antibodies. Antibody preparations can be simply serum from an immunized animal, or if desired, polyclonal antibodies can be isolated from the serum by, for example, affinity chromatography using immobilized immunogen.

To produce monoclonal antibodies, antibody-producing splenocytes can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, Nature, 256: 495-497 (1975)), the human B-cell hybridoma technique (Kozbar et al., Immunology Today, 4: 72 (1983)), and the EBV-hybridoma technique to produce human monoclonal antibodies (Cole et al., Monoclonal Antibodies and Cancer Therapy, Alan R. Liss, Inc. pp. 77-96 (1985)). Hybridoma cells can be screened immunochemically for production of antibodies that are specifically reactive with C-terminal APP fragment or tau peptide, or polypeptide, and monoclonal antibodies isolated from the media of a culture comprising such hybridoma cells.

II. Preparation of Primary Cell Cultures from Transgenic Drosophila

Cells can be obtained from any transgenic fly of the invention and maintained in primary culture where their phenotype can be evaluated or where they can be used to identify agents active in neurodegeneration. Any technique known in the art can be applied to the isolation of cells from a transgenic fly, to their culture, to optionally promoting their differentiation, and to study their phenotype.

In brief, one or more cells are obtained from a transgenic fly, at any developmental stage, by dissection and/or dissociation of organs or tissues of the fly. The cells are placed into a culture medium compatible with maintaining their viability long enough to study their phenotype, typically for several hours to several days or weeks. A variety of techniques are available for dissociation of fly tissues. For example, several embryos can be collectively homogenized using a loose fitting Potter-Elvehjem glass homogenizer to provide a whole embryo cell suspension. The cells can be homogenized and cultured in a sterile medium such as Schneider's Incomplete Medium (Gibco-BRL, Gaithersburg, Md.) supplemented with 10% non-heat inactivated fetal bovine serum and maintained without CO₂ in an incubator at a temperature in the range 20-30° C. See, e.g., Guha et al., J. Cell Sci. 116:3373-86 (2003). Individual cell types can be selected from the culture, e.g., by cell morphology or other characteristics. For example, whole animal cell sorting can be used to isolated desired cell types, e.g., neuronal precursor cells or hemocytes, based on genotype using, for example, lacZ expression (see Krasnow et al., Science 251:81-85 (1991)) or expression of a GFP-tagged protein (see Guha et al., J. Cell Sci. 116:3373-86 (2003)).

Cells in primary cultures can differentiate into a variety of terminally differentiated cells including nerve and muscle (Hayashi et al., In Vitro Cell Dev Biol Anim 30A:202-8 (1994)) or nerve and epidermis (Lüer et al., Development 116:377-85 (1992)). Cells also can be cultured from later developmental stages or adults. For example, eye imaginal discs can be dissociated from larvae or pupae, and used to study neurite outgrowth if taken from pupae, or used to provide mitotic cells from earlier stages (Li et al., J Neurobiol 28:363-80 (1995)). Cells in primary culture can be studied soon after removal, i.e., at the developmental stage when removed, or allowed to differentiate in vitro and studied subsequent to differentiation.

The phenotype of cultured cells from transgenic flies containing CTFAPP or CTFAPP and tau can be used to study the effect of these polypeptides on phenomena related to neurodegeneration. For example, cultured cells can be examined for the production of extracellular amyloid plaques or intracellular tangles, for example, by examining the distribution of labeled antibodies that specifically bind either CTFAPP, Aβ or tau. Alterations to the organization of the cytoskeleton can be investigated with labeled antibodies to cytoskeletal proteins such as actin, tubulin, and associated proteins. Neurological function can be studied at the cellular level by performing electrophysiological measurements of ion channel activity, or release of neurotransmitters. Cell-cell associations and gene expression can also provide clues to pathological mechanisms active in neurodegenerative diseases such as Alzheimer's disease. Furthermore, primary cultures can be employed in a screening process to identify chemical agents that are likely to have a beneficial effect on neurodegenerative disease, by examining the effect of candidate agents on transgenic cell phenotype.

III. Molecular Techniques

In the present invention, DNA sequences that encode tau or human Aβ42_{Italian} are cloned into transformation vectors suitable for the generation of transgenic flies.

Generation of DNA Sequences Encoding Tau or Human C-Terminal APP Fragment

DNA sequences encoding tau and C-terminal APP fragment can be obtained from genomic DNA or be generated by synthetic means using methods well known in the art (Sambrook et al., Molecular Biology: A laboratory Approach, Cold Spring Harbor, N.Y. (1989); Ausubel, et al., Current protocols in Molecular Biology, Greene Publishing, Y, (1995)). Briefly, human genomic DNA can be isolated from peripheral blood or mucosal scrapings by phenol extraction, or by extraction with kits such as the QIAamp Tissue kit (Qiagen, Chatsworth, Cal.), Wizard genomic DNA purification kit (Promega, Madison, Wis.), and the ASAP genomic DNA isolation kit (Boehringer Mannheim, Indianapolis, Ind.). DNA sequences encoding tau and C-terminal APP fragment can then be amplified from genomic DNA by polymerase chain reaction (PCR) (Mullis and Faloona Methods Enzymol., 155: 335 (1987)), herein incorporated by reference) and cloned into a suitable recombinant cloning vector.

Alternatively, a cDNA that encodes tau or human C-terminal APP fragment can be amplified from mRNA using RT-PCR and cloned into a suitable recombinant cloning vector. RNA may be prepared by any number of methods known in the art; the choice may depend on the source of the sample. Methods for preparing RNA are described in Davis et al., Basic Methods in Molecular Biology, Elsevier, N.Y., Chapter 11 (1986); Ausubel et al., Current Protocols in Molecular Biology, Chapter 4, John Wiley and Sons, NY (1987); Kawasaki and Wang, PCR Technology, ed. Erlich, Stockton Press NY (1989); Kawasaki, PCR Protocols: A Guide to Methods and Applications, Innis et al. eds. Academic Press, San Diego (1990); all of which are incorporated herein by reference.

Following generation of sequences that encode tau or CTFAPP fragment by PCR or RT-PCR, the sequences preferably are cloned into an appropriate sequencing vector in order that the sequence of the cloned fragment can be confirmed by nucleic acid sequencing in both directions.

Suitable recombinant cloning vectors for use in the present invention contain nucleic acid sequences that enable the vector to replicate in one or more selected host cells. Typically in cloning vectors, this sequence is one that enables the vector to replicate independently of the host chromosomal DNA and includes origins of replication or autonomously replicating sequences. Such sequences are well known for a variety of bacteria, yeast and viruses. For example, the origin of replication from the plasmid pBR322 is suitable for most Gram-negative bacteria, the 2 micron plasmid origin is suitable for yeast, and various viral origins (e.g. SV40, adenovirus) are useful for cloning vectors in mammalian cells. Generally, the origin of replication is not needed for mammalian expression vectors unless these are used in mammalian cells able to replicate high levels of DNA, such as COS cells.

Advantageously, a cloning or expression vector may contain a selection gene also referred to as a selectable marker. This gene encodes a protein necessary for the survival or growth of transformed host cells grown in a selective culture medium. Host cells not transformed with the vector containing the selection gene will therefore not survive in the culture medium. Typical selection genes encode proteins that confer resistance to antibiotics and other toxins, e.g. ampicillin, neomycin, methotrexate or tetracycline, complement auxotrophic deficiencies, or supply critical nutrients not available in the growth media.

Since cloning is most conveniently performed in E. coli, an E. coli-selectable marker, for example, the β-lactamase gene that confers resistance to the antibiotic ampicillin, is of use. These can be obtained from E. coli plasmids, such as pBR322 or a pUC plasmid such as pUC18 orpUC19.

Sequences that encode tau or human C-terminal APP fragment can also be directly cloned into a transformation vector suitable for generation of transgenic fly such as vectors that allow for the insertion of sequences in between transposable elements, or insertion downstream of an UAS element, such as pUAST. Vectors suitable for the generation of transgenic flies preferably contain marker genes such that the transgenic fly can be identified such as, the white gene, the rosy gene, the yellow gene, the forked gene, and others mentioned previously. Suitable vectors can also contain tissue specific control sequences as described earlier, such as, the sevenless promoter/enhancer, the eyeless promoter/enhancer, glass-responsive promoters (GMR)/enhancers useful for expression in the eye; and enhancers/promoters derived from the dpp or vestigial genes useful for expression in the wing.

Sequences that encode tau or human C-terminal APP fragment are ligated into a recombinant vector in such a way that the expression control sequences are operatively linked to the coding sequence.

Herein, DNA sequences that encode tau or human C-terminal APP fragment can be generated through the use of polymerase chain reaction (PCR), or RT-PCR which uses RNA-directed DNA polymerase (e.g., reverse transcriptase) to synthesize cDNAs which is then used for PCR.

IV. Phenotypes and Methods of Detecting Altered Phenotypes

Any observable and/or measurable physical or biochemical characteristic of a fly is a phenotype that can be assessed according to the present invention. Transgenic flies with desired properties, for example, properties appropriate for screening assays, can be produced by identifying flies that exhibit an altered phenotype as compared to control (e.g., wild-type flies, or flies in which the transgene is not expressed). Therapeutic agents can be identified by screening for agents, that upon administration, result in a change in an altered phenotype of the transgenic fly as compared to a transgenic fly that has not been administered a candidate agent.

A change in an altered phenotype includes either complete or partial reversion of the phenotype observed. Complete reversion is defined as the absence of the altered phenotype, or as 100% reversion of the phenotype to that phenotype observed in control flies. Partial reversion of an altered phenotype can be 5%, 10%, 20%, preferably 30%, more preferably 50%, and most preferably greater than 50% reversion to that phenotype observed in control flies. Example measurable parameters include, but are not limited to, morphology of organs, such as the eye; distribution of tissues and organs; behavioral phenotypes (such as, appetite and mating); and locomotor ability.

In some embodiments only a single phenotype is determined. In other embodiments two or more phenotypes are determined. In yet other embodiments, multiple phenotypes (traits), e.g., 2, 3, 4, 5, 7, 10, or more traits are determined and used to create a “phenoprofile.” The traits measured can be solely movement traits, solely behavioral traits, solely morphological traits, or a mixture of traits in multiple categories. In some embodiments at least one movement trait and at least one non-movement trait is assessed. Phenotypes or phenoprofiles can be compared for individual flies or for populations of flies. If a population of flies is being studied, global values for each trait can be compared and a subset of traits that differs significantly between the populations can be identified. The subset of traits and the values of the traits for a particular population (e.g., the parental fly stock) is referred to as a “phenoprint” of that population. Thus, the traits in which a test population of biological specimens differs from a population of control biological specimens is referred to as the “phenoprint” of the test population.

For each of the various trait parameters described, statistical measures can be determined. See, for example, PRINCIPLES OF BIOSTATISTICS, second edition (2000) Mascello et al., Duxbury Press. Examples of statistics per trait parameter include distribution, mean, variance, standard deviation, standard error, maximum, minimum, frequency, latency to first occurrence, latency to last occurrence, total duration (seconds or %), mean duration (if relevant).

Locomotor Phenotypes

Locomotor phenotypes can be assessed, for example, as described in U.S. Application Nos. 2004/0076583, 2004/0076318, and 2004/0076999, each of which is hereby incorporated by reference in its entirety. For example, locomotor ability can be assessed in a climbing assay by placing flies in a vial, knocking them to the bottom of the vial, then counting the number of flies that climb past a given mark on the vial during a defined period of time. In this example, 100% locomotor activity of control flies is represented by the number of flies that climb past the given mark, while flies with an altered locomotor activity can have 80%, 70%, 60%, 50%, preferably less than 50%, or more preferably less than 30% of the activity observed in a control fly population.

In one aspect, the traits are measured by detecting and serially analyzing the movement of a population of flies in containers, e.g., vials. Movement of the flies can be monitored by a recording instrument, such as a CCD-video camera, the resultant images can be digitized, analyzed using processor-assisted algorithms as described herein, and the analysis data stored in a computer-accessible manner. For example, in measuring traits related to fly movement, the trajectory of each animal may be monitored by calculation of one or more variables (e.g., speed, vertical only speed, vertical distance, turning frequency, frequency of small movements, etc.) for the animal. Values of such a variable are then averaged for population of animals in the vial and a global value is obtained describing the trait for each population (e.g., parental stock flies and transgenic flies).

“Movement trait data” as used herein refers to the measurements made of one or more movement traits. Examples of “movement trait data” measurements include, but are not limited to X-pos, X-speed, speed, turning, stumbling, size, T-count, P-count, T-length, Crosshigh, Crosslow, and F-count. Descriptions of these particular measurements are provided below.

Examples of such “movement traits” include, but are not limited to:

a) total distance (average total distance traveled over a defined period of time);

b) X only distance (average distance traveled in X direction over a defined period of time;

c) Y only distance (average distance traveled in Y direction over a defined period of time);

d) average speed (average total distance moved per time unit);

e) average X-only speed (distance moved in X direction per time unit);

f) average Y-only speed (distance moved in Y direction per time unit);

g) acceleration (the rate of change of velocity with respect to time);

h) turning;

i) stumbling;

j) spatial position of one fly to a particular defined area or point (examples of spatial position traits include (1) average time spent within a zone of interest (e.g., time spent in bottom, center, or top of a container; number of visits to a defined zone within container); (2) average distance between a fly and a point of interest (e.g., the center of a zone); (3) average length of the vector connecting two sample points (e.g., the line distance between two flies or between a fly and a defined point or object); (4) average time the length of the vector connecting the two sample points is less than, greater than, or equal to a user define parameter; and the like);

k) path shape of the moving fly, i.e., a geometrical shape of the path traveled by the fly (examples of path shape traits include the following: (1) angular velocity (average speed of change in direction of movement); (2) turning (angle between the movement vectors of two consecutive sample intervals); (3) frequency of turning (average amount of turning per unit of time); (4) stumbling or meandering (change in direction of movement relative to the distance); and the like. This is different from stumbling as defined above. Turning parameters may include smooth movements in turning (as defined by small degrees rotated) and/or rough movements in turning (as defined by large degrees rotated).

Movement traits can be quantified, for example, using the following parameters:

X-Pos: The X-Pos score is calculated by concatenating the lists of x-positions for all trajectories and then computing the average of all values in the concatenated list.

X-Speed: The X-Speed score is calculated by first computing the lengths of the x-components of the speed vectors by taking the absolute difference in x-positions for subsequent frames. The resulting lists of x-speeds for all trajectories are then concatenated and the average x-speed for the concatenated list is computed.

Speed: The Speed score is calculated in the same way as the X-Speed score, but instead of only using the length of the x-component of the speed vector, the length of the whole vector is used. That is, [length]=square root of ([x-length]²+[y-length]²).

Turning: The Turning score is calculated in the same way as the Speed score, but instead of using the length of the speed vector, the absolute angle between the current speed vector and the previous one is used, giving a value between 0 and 90 degrees.

Stumbling: The Stumbling score is calculated in the same way as the Speed score, but instead of using the length of the speed vector, the absolute angle between the current speed vector and the direction of body orientation is used, giving a value between 0 and 90 degrees.

Size: The Size score is calculated in the same way as the Speed score, but instead of using the length of the speed vector, the size of the detected fly is used.

T-Count: The T-Count score is the number of trajectories detected in the movie.

P-Count: The P-Count score is the total number of points in the movie (i.e., the number of points in each trajectory, summed over all trajectories in the movie).

T-Length: The T-Length score is the sum of the lengths of all speed vectors in the movie, giving the total length all flies in the movie have walked.

Crosshigh: The Crosshigh score is the number of trajectories that either crossed the line set at a value in the negative x-direction (from bottom to top of the vial) in the upper half of the vial during the movie, or that were already above that line at the start of the movie. The latter criterion was included to compensate for the fact that flies sometimes don't fall to the bottom of the tube. In other words this score measures the number of detected flies that either managed to hold on to the tube or that managed to climb above the x line within the length of the movie.

Crosslow: The Crosslow score is equivalent to the Crosshigh score, but uses a line in the lower half of the vial instead.

F-Count: The F-Count score counts the number of detected flies in each individual frame, and then takes the maximum of these values over all frames. It thereby measures the maximum number of flies that were simultaneously visible in any single frame during the movie.

LIP: Measures the number of flies which “stall” (i.e. move very little for a certain period of time specified by the user) in a video

maxheight: The maximum sum of x-positions over all frames, divided by the number of flies in the vials as estimated by the fly count metric.

Flycount: Same as the fcount metric, except that the 97^{th} percentile is used instead of the maximum.

fcross at t: The number of flies that were simulatenously above a set height within the specified time frame (t).

The assignment of directions in the X-Y coordinate system is arbitrary. For purposes of this disclosure, “X” refers to the vertical direction (typically along the long axis of the container in which the flies are kept) and “Y” refers to movement in the horizontal direction (e.g., along the surface of the vial).

Eye Phenotypes

A double transgenic fly according to the invention can exhibit an altered eye phenotype, caused by progressive neurodegeneration in the eye that leads to measurable morphological changes in the eye (Femandez-Funez et al., Nature 408:101-106 (2000); Steffan et. al, Nature 413:739-743 (2001)). The Drosophila eye is composed of a regular trapezoidal arrangement of eight rhabdomeres (seven visible in a single section) produced by the photoreceptor neurons of each Drosophila ommatidium. A phenotypic eye mutant according to the invention leads to a progressive loss of rhabdomeres and subsequently a rough-textured eye. A rough textured eye phenotype is easily observed by microscope or video camera. In a screening assay for compounds which alter this phenotype, one may observe slowing of the photoreceptor degeneration and improvement of the rough-eye phenotype (Steffan et. al, Nature 413:739-743 (2001)).

Behavioral Phenotypes

Neuronal degeneration in the central nervous system will give rise to behavioral deficits, including but not limited to locomotor deficits, that can be assayed and quantitated in both larvae and adult Drosophila. For example, failure of Drosophila adult animals to climb in a standard climbing assay (see, e.g. Ganetzky and Flannagan, J. Exp. Gerontology 13:189-196 (1978); LeBourg and Lints, J. Gerontology 28:59-64 (1992)) is quantifiable, and indicative of the degree to which the animals have a motor deficit and neurodegeneration. Neurodegenerative phenotypes include, but are not limited to, progressive loss of neuromuscular control, e.g. of the wings; progressive degeneration of general coordination; progressive degeneration of locomotion, and progressive loss of appetite. Other aspects of fly behavior that can be assayed include but are not limited to circadian behavioral rhythms, feeding behaviors, inhabituation to external stimuli, and odorant conditioning. All of these phenotypes are measured by one skilled in the art by standard visual observation of the fly.

Another neural degeneration phenotype, is a reduced life span, for example, the Drosophila life span can be reduced by 10-80%, e.g., approximately, 30%, 40%, 50%, 60%, or 70%.

Memory and Learning Phenotypes

In Drosophila, the best characterized assay for associative learning and memory is an odor-avoidance behavioral task (T. Tully, et al. J. Comp. Physiol. A157, 263-277 (1985), incorporated herein by reference). This classical (Pavlovian) conditioning involves exposing the flies to two odors (the conditioned stimuli, or CS), one at a time, in succession. During one of these odor exposures (the CS+), the flies are simultaneously subjected to electric shock (the unconditioned stimulus, or US), whereas exposure to the other odor (the CS−) lacks this negative reinforcement. Following training, the flies are then placed at a “choice point,” where the odors come from opposite directions, and expected to decide which odor to avoid. By convention, learning is defined as the fly's performance when testing occurs immediately after training. A single training trial produces strong learning: a typical response is that >90% of the flies avoid the CS+. Performance of wild-type flies from this single-cycle training decays over a roughly 24-hour period until flies once again distribute evenly between the two odors. Flies can also form long-lasting associative olfactory memories, but normally this requires repetitive training regimens.

V. Utility of Transgenic Flies

Disease Model

The transgenic flies of the invention provide a model for neurodegeneration as is found in human neurological diseases such as Alzheimer's and tauopathies, such as amyotrophic lateral sclerosis/parkinsonism-dementia complex of Guam argyrophilic grain dementia, corticobasal degeneration, dementia pugilistica, diffuse neurofibrillary tangles with calcification, frontotemporal dementia with Parkinsonism linked to chromosome 17 (FTDP-17), Pick's disease, progressive subcortical gliosis, progressive supranuclear palsy (PSP), tangle only dementia, Creutzfeldt-Jakob disease, Down syndrome, Gerstmann-Sträussler-Scheinker disease, Hallervorden-Spatz disease, myotonic dystrophy, age-related memory impairment, Alzheimer's disease, amyotrophic lateral sclerosis, amyotrophic lateral/parkinsonism-dementia complex of Guam, auto-immune conditions (eg Guillain-Barre syndrome, Lupus), Biswanger's disease, brain and spinal tumors (including neurofibromatosis), cerebral amyloid angiopathies (Journal of Alzheimer's Disease vol. 3, 65-73 (2001)), cerebral palsy, chronic fatigue syndrome, Creutzfeldt-Jacob disease (including variant form), corticobasal degeneration, conditions due to developmental dysfunction of the CNS parenchyma, conditions due to developmental dysfunction of the cerebrovasculature, dementia—multi infarct, dementia—subcortical, dementia with Lewy bodies, dementia of human immunodeficiency virus (HIV), dementia lacking distinct histology, dendatorubopallidolusian atrophy, diseases of the eye, ear and vestibular systems involving neurodegeneration (including macular degeneration and glaucoma), Down's syndrome, dyskinesias (paroxysmal), dystonias, essential tremor, Fahr's syndrome, Friedrich's ataxia, fronto-temporal dementia and Parkinsonism linked to chromosome 17 (FTDP-17), frontotemporal lobar degeneration, frontal lobe dementia, hepatic encephalopathy, hereditary spastic paraplegia, Huntington's disease, hydrocephalus, pseudotumor cerebri and other conditions involving CSF dysfunction, Gaucher's disease, spinal muscular atrophy (Hirayama disease, Werdnig-Hoffman disease, Kugelberg-Welander disease), Korsakoff's syndrome, Machado-Joseph disease, mild cognitive impairment, monomelic amyotrophy, motor neuron diseases, multiple system atrophy, multiple sclerosis and other demyelinating conditions (eg leukodystrophies), myalgic encephalomyelitis, myotonic dystrophy, myoclonus neurodegeneration induced by chemicals, drugs and toxins, neurological manifestations of AIDS including AIDS dementia, neurological conditions (any) arising from polyglutamine expansions, neurological/cognitive manifestations and consequences of bacterial and/or virus infections, including but not restricted to enteroviruses, Niemann-Pick disease, non-Guamanian motor neuron disease with neurofibrillary tangles, non-ketotic hyperglycinemia, olivo-ponto cerebellar atrophy, opthalmic and otic conditions involving neurodegeneration, including macular degeneration and glaucoma, Parkinson's disease, Pick's disease, polio myelitis including non-paralytic polio, primary lateral sclerosis, prion diseases including Creutzfeldt-Jakob disease, kuru, fatal familial insomnia, and Gerstmann-Straussler-Scheinker disease, prion protein cerebral amyloid angiopathy, postencephalitic Parkinsonism, post-polio syndrome, prion protein cerebral amyloid angiopathy, progressive muscular atrophy, progressive bulbar palsy, progressive supranuclear palsy, restless leg syndrome, Rett syndrome, Sandhoff disease, spasticity, spino-bulbar muscular atrophy (Kennedy's disease), spinocerebellar ataxias, sporadic fronto-temporal dementias, striatonigral degeneration, subacute sclerosing panencephalitis, sulphite oxidase deficiency, Sydenham's chorea, tangle only dementia, Tay-Sach's disease, Tourette's syndrome, transmissable spongiform encephalopathies, vascular dementia, and Wilson disease.

Methods for Identifying Therapeutic Agents

The present invention further provides a method for identifying a therapeutic agent for neurodegenerative disease using the transgenic flies disclosed herein. As used herein, a “therapeutic agent” refers to an agent that ameliorates the symptoms of neurodegenerative disease as determined by a physician. For example, a therapeutic agent can reduce one or more symptoms of neurodegenerative disease, delay onset of one or more symptoms, or prevent, or cure.

To screen for a therapeutic agent effective against a neurodegenerative disorder such as disease, a candidate agent is administered to a transgenic fly. The transgenic fly is then assayed for a change in the phenotype as compared to the phenotype displayed by a control transgenic fly that has not been administered a candidate agent. An observed change in phenotype is indicative of an agent that is useful for the treatment of disease.

A candidate agent can be administered by a variety of means. For example, an agent can be administered by applying the candidate agent to the fly culture media, for example by mixing the agent in fly food, such as a yeast paste that can be added to fly cultures. Alternatively, the candidate agent can be prepared in a 1% sucrose solution, and the solution fed to the flies for a specified time, such as 10 hours, 12 hours, 24 hours, 48 hours, or 72 hours. In one embodiment, the candidate agent is microinjected into the fly's hemolymph, as described in WO 00/37938, published Jun. 29, 2000. Other modes of administration include aerosol delivery, for example, by vaporization of the candidate agent.

The candidate agent can be administered at any stage of fly development including fertilized eggs, embryonic, larval and adult stages. In a preferred embodiment, the candidate agent is administered to an adult fly. More preferably, the candidate agent is administered during a larval stage, for example by adding the agent to the fly culture at the third larval instar stage, which is the main larval stage in which eye development takes place.

The agent can be administered in a single dose or multiple doses. Appropriate concentrations can be determined by one skilled in the art, and will depend upon the biological and chemical properties of the agent, as well as the method of administration. For example, concentrations of candidate agents can range from 0.0001 μM to 20 mM when delivered orally or through injection, 0.1 μM to 20 mM, 1 μM-10 mM, or 10 μM to 5 mM.

For efficiency of screening the candidate agents, in addition to screening with individual candidate agents, the candidate agents can be administered as a mixture or population of agents, for example a library of agents. As used herein, a “library” of agents is characterized by a mixture more than 20, 100, 10³, 10⁴, 10⁵, 10⁶, 10⁸, 10¹², or 10¹⁵ individual agents. A “population of agents” can be a library or a smaller population such as, a mixture less than 3, 5, 10, or 20 agents. A population of agents can be administered to the transgenic flies and the flies can be screened for complete or partial reversion of a phenotype exhibited by the transgenic flies. When a population of agents results in a change of the transgenic fly phenotype, individual agents of the population can then be assayed independently to identify the particular agent of interest.

In a preferred embodiment, a high throughput screen of candidate agents is performed in which a large number of agents, at least 50 agents, 100 agents or more are tested individually in parallel on a plurality of fly populations. A fly population contains at least 2, 10, 20, 50, 100, or more adult flies or larvae. In one embodiment, locomotor phenotypes, behavioral phenotypes (e.g. appetite, mating behavior, and/or life span), or morphological phenotypes (e.g., shape size, or location of a cell, or organ, or appendage; or size shape, or growth rate of the fly) are observed by creating a digitized movie of the flies in the population and the movie is analyzed for fly phenotype.

Candidate Agents

Agents that are useful in the screening assays of the present inventions include biological or chemical compounds that when administered to a transgenic fly have the potential to modify an altered phenotype, e.g. partial or complete reversion of the phenotype. Agents include any recombinant, modified or natural nucleic acid molecule; a library of recombinant, modified or natural nucleic acid molecules; synthetic, modified or natural peptides; a library of synthetic, modified or natural peptides; organic or inorganic compounds; or a library of organic or inorganic compounds, including small molecules. Agents can also be linked to a common or unique tag, which can facilitate recovery of the therapeutic agent.

Example agent sources include, but are not limited to, random peptide libraries as well as combinatorial chemistry-derived molecular library made of D- and/or L-configuration amino acids; phosphopeptides (including, but not limited to, members of random or partially degenerate, directed phosphopeptide libraries; see, e.g., Songyang et al., Cell 72:767-778 (1993)); antibodies (including, but not limited to, polyclonal, monoclonal, humanized, anti-idiotypic, chimeric or single chain antibodies, and FAb, F(ab′)2 and FAb expression library fragments, and epitope-binding fragments thereof); and small organic or inorganic molecules.

Many libraries are known in the art that can be used, e.g. chemically synthesized libraries, recombinant libraries (e.g., produced by phage), and in vitro translation-based libraries. Examples of chemically synthesized libraries are described in Fodor et al., Science 251:767-773 (1991); Houghten et al., Nature 354:84-86 (1991); Lam et al., Nature 354:82-84 (1991); Medyuski, Bio/Technology 12:709-710 (1994); Gallop et al., J. Medicinal Chemistry 37:1233-1251 (1994); Ohlmeyer et al., Proc. Natl. Acad. Sci. USA 90: 10922-10926 (1993); Erb et al., Proc. Natl. Acad. Sci. USA 91:11422-11426 (1994); Houghten et al., Biotechniques 13:412 (1992); Jayawickreme et al., Proc. Natl. Acad. Sci. USA 91:1614-1618 (1994); Salmon et al., Proc. Natl. Acad. Sci. USA 90:11708-11712 (1993); PCT Publication No. WO 93/20242; and Brenner and Lerner, Proc. Natl. Acad. Sci. USA 89:5381-5383 (1992). By way of examples of nonpeptide libraries, a benzodiazopine library (see e.g., Bunin et al., Proc. Natl. Acad. Sci. USA 91:4708-4712 (1994)) can be adapted for use.

Peptoid libraries (Simon et al., Proc. Natl. Acad. Sci. USA 89:9367-9371 (1992)) can also be used. Another example of a library that can be used, in which the amide functionalities in peptides have been permethylated to generate a chemically transformed combinatorial library, is described by Ostresh et al. Proc. Natl. Acad. Sci. USA 91:11138-11142 (1994). Examples of phage display libraries wherein peptide libraries can be produced are described in Scott & Smith, Science 249:386-390 (1990); Devlin et al., Science, 249:404-406 (1990); Christian et al., J. Mol. Biol. 227:711-718 (1992); Lenska, J. Immunol. Meth. 152:149-157 (1992); Kay et al., Gene 128:59-65 (1993); and PCT Publication No. WO 94/18318 dated Aug. 18, 1994.

Agents that can be tested and identified by methods described herein can include, but are not limited to, compounds obtained from any commercial source, including Aldrich (Milwaukee, Wis. 53233), Sigma Chemical (St. Louis, Mo.), Fluka Chemie AG (Buchs, Switzerland) Fluka Chemical Corp. (Ronkonkoma, N.Y.;), Eastman Chemical Company, Fine Chemicals (Kingsport, Tenn.), Boehringer Mannheim GmbH (Mannheim, 25 Germany), Takasago (Rockleigh, N.J.), SST Corporation (Clifton, N.J.), Ferro (Zachary, La. 70791), Riedel-deHaen Aktiengesellschaft (Seelze, Germany), PPG Industries Inc., Fine Chemicals (Pittsburgh, Pa. 15272). Further any kind of natural products may be screened using the methods described herein, including microbial, fungal, plant or animal extracts.

Furthermore, diversity libraries of test agents, including small molecule test compounds, may be utilized. For example, libraries may be commercially obtained from Specs and BioSpecs B.V. (Rijswijk, The Netherlands), Chembridge Corporation (San Diego, Calif.), Contract Service Company (Dolgoprudoy, Moscow Region, Russia), Comgenex USA Inc. (Princeton, N.J.), Maybridge Chemicals Ltd. (Cornwall PL34 OHW, United Kingdom), and Asinex (Moscow, Russia).

Still further, combinatorial library methods known in the art, can be utilized, including, but not limited to: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other approaches are applicable to peptide, non-peptide oligomer or small molecule libraries of compounds (Lam, Anticancer Drug Des.12: 145 (1997)). Combinatorial libraries of test compounds, including small molecule test compounds, can be utilized, and may, for example, be generated as disclosed in Eichler & Houghten, Mol. Med. Today 1:174-180 (1995); Dolle, Mol. Divers. 2:223-236 (1997); and Lam, Anticancer Drug Des. 12:145-167 (1997).

Examples of methods for the synthesis of molecular libraries can be found in the art, for example in: DeWitt et al., Proc. Natl. Acad. Sci. USA 90:6909 (1993); Erb et al., Proc. Natl. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33:2059 (1994); Carell et al., Angew. Chem. Int. Ed. Engl. 33:2061 (1994); and Gallop et al., 15 J. Med. Chem. 37:1233 (1994).

A library of agents can also be a library of nucleic acid molecules; DNA, RNA, or analogs thereof. For example, a cDNA library can be constructed from mRNA collected from a cell, tissue, organ or organism of interest, or genomic DNA can be treated to produce appropriately sized fragments using restriction endonucleases or methods that randomly fragment genomic DNA. A library containing RNA molecules can be constructed, for example, by collecting RNA from cells or by synthesizing the RNA molecules chemically. Diverse libraries of nucleic acid molecules can be made using solid phase synthesis, which facilitates the production of randomized regions in the molecules. If desired, the randomization can be biased to produce a library of nucleic acid molecules containing particular percentages of one or more nucleotides at a position in the molecule (U.S. Pat. No. 5,270,163).

Methods for Identifying Genetic Modifiers

The transgenic flies described herein can be used to identify genes that affect neurodegenerative diseases such as AD. Such genes, termed “genetic modifiers,” can be identified through their ability to alter a phenotype produced by one or more transgenes, such as CTFAPP or CTFAPP plus tau. For example, a collection of flies harboring mutations in genes which are candidate genetic modifiers can be crossed with either a single transgenic fly line expressing CTFAPP or a double transgenic fly line expressing CTFAPP plus tau. If a given mutation alters the phenotype associated with the transgene or combination of transgenes, then the gene having that mutation is identified as a genetic modifier. For example, a transgenic fly expressing CTFAPP and showing an eye phenotype (e.g., altered ommatidial packing), can be crossed with mutant flies, and genetic modifiers identified by rescue of the normal eye phenotype. Any phenotype, preferably a visible phenotype, can be used for screening to identify genetic modifiers. The transgene or combination of transgenes can be directly driven by a promoter or driven by the UAS/GAL4 system. Once a mutant strain containing a genetic modifier is identified, the locus of the modifier can be determined using established techniques, such as deficiency mapping (Parks et al., Nat. Genet. 36:288-92 (2004)) or meiotic recombination mapping in concert with single nucleotide polymorphisms (Hoskins et al., Genome Res. 11:1100-13 (2001)). A transgenic strain harboring a mutation in a genetic modifier can also form the basis of a high throughput screen, using techniques outlined above, to identify drugs which modify the interaction between the genetic modifier and the transgene or combination of transgenes.

### EXAMPLES

### Example 1

### Generation of Transgenic Drosophila Expressing CTFAPP

A transgenic strain of Drosophila melanogaster was prepared containing the CTFAPP fragment of human APP695. Flies were injected with a construct containing the SP65/A4CT vector of Dyrks et al. (FEBS Lett. 309, 20-24 (1992)) containing cDNA encoding amino acids 596-695 of human APP695 fused in frame to the signal peptide of APP695, with an upstream activating sequence (UAS) placed upstream of the SP65/A4CT vector. The transgenic strain was then crossed with a with a GMR-GAL4 driver strain of D. melanogaster, resulting in expression of CTFAPP in the eye.

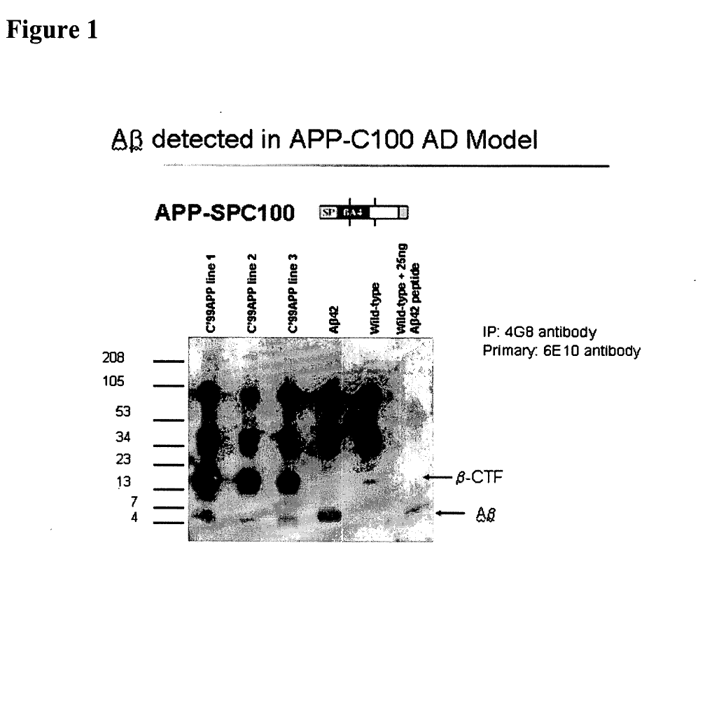

FIG. 1 shows a Western blot of extracts from the CTFAPP transgenic flies demonstrating the production of Aβ42 in the CTFAPP transgenic flies. The immuniprecipitation antibody was antibody 4G8 (Signet) which is specific for human APP/Amyloid β. The primary antibody for the Western blot was 6E10 (Signet), also specific to human APP/Amyloid β, although having a slightly different epitope than 4G8. The secondary antibody was an HRP conjugated goat-anti-mouse. Three separate transgenic lines generated by the procedure described above are shown in the first three lanes from the left. The A042 control flies shown in the fourth lane from the left were obtained from an Aβ42 transgenic strain obtained by crossing a transgenic D. melanogaster strain containing cDNA encoding Aβ42 fused in frame to an argos signal sequence with a D. melanogaster GMR-GAL4 driver strain. Wild type fly extract presented as a control (lane 5) did not express A042.

### Example 2

### Generation of Transgenic Drosophila Expressing CTFAPP and tau

A transgenic Drosophila melanogaster strain containing a transgene encoding human tau and a transgenic Drosophila melanogaster strain containing a transgene encoding human CTFAPP are generated as described herein. The two transgenic fly strains are then recombined to obtain a double transgenic Drosophila melanogaster strain containing genes encoding both human tau and human CTFAPP.

Transgene Constructs

The UAS/GAL4 system is used to generate both the CTFAPP and tau transgenic flies. A cDNA encoding the longest human brain tau isoform is cloned using standard ligation techniques (Sambrook et al., Molecular Biology: A laboratory Approach, Cold Spring Harbor, N.Y. 1989) into vector pUAST (Brand and Perrimon, Development 118:401-415 (1993)) as an EcoRI fragment in order to generate transformation vector, pUAS-_{2N4R}tauwt. The tau isoform, which is represented by SEQ ID NO:15 (amino acid sequence) and SEQ ID NO:16 (nucleic acid sequence), contains tau exons 2 and 3 as well as four microtubule-binding repeats.

A pExP-UAS transformation vector carrying DNA sequence encoding CTFAPP is generated. The vector encodes CTFAPP fused to the human APP signal peptide and a myc tag (pExP-UAS-CTFAPP-I, sequence shown in SEQ ID NO:17). To generate pExP-UAS-CTFAPP-I, a DNA sequence encoding CTFAPP is first fused in frame to a synthetic oligonucleotide encoding the myc tag. This DNA fragment is then fused in frame to a PCR amplified sequence encoding the human APP signal sequence. The resulting DNA sequence is then cloned into the pExP-UAS vector.

Transgenic Strains

To generate transgenic Drosophila lines expressing either tau or CTFAPP, the pUAST or pExP constructs described above, either pExP-UAS-CTFAPP-I or pUAS-_{2N4R}tauwt is injected into a y¹ w¹¹¹⁸ or w¹¹¹⁸ Drosophila melanogaster embryo as described in Rubin and Spradling (Science 218:348-353, (1982)).

In the case of pUAS-_{2N4R}tau-wt, six transgenic lines are generated and classified by visual inspection, as described herein, as strong, medium, or weak based on the severity of the eye phenotype observed after crossing with a GMR-GAL4 driver strain.

In the case of pExP-UAS-CTFAPP-I, transgenic lines are generated and classified as strong, medium, or weak based on the severity of the eye phenotype observed after crossing with a GMR-GAL4 driver strain. Transgenic Drosophila strains of moderate eye phenotype that carry the GMR-GAL4 driver and pExP-UAS-CTFAPP-I or pUAS-_{2N4R}tauwt are then crossed to generate a double transgenic Drosophila line that express both tau and CTFAPP peptide. Crossing the single transgenic flies of moderate eye phenotype results in a synergistic eye phenotype classified as strong.

In the case of transformation constructs pExP-UAS-:CTFAPP-I and pUAS-_{2N4R}tauwt, transgenic lines are generated by injecting the construct into a y¹ w¹¹¹⁸ or w¹¹¹⁸ Drosophila melanogaster embryo as described in (Rubin and Spradling, Science 218:348-353 (1982)) and screened for the insertion of transgene into genomic DNA by monitoring eye color. The pExP and pUAST vectors carry a portion of the white gene marker. Transgenic Drosophila carrying CTFAPP transgene are then crossed with elav-GAL4 driver strains for expression of the transgene in the central nervous system. If the crosses do not result in a measurable phenotype, the transgene is mobilized for expansion of copy number by crossing transgenic Drosophila carrying CTFAPP transgene with Drosophila that carry a source of transposase. Remobilization of the transposable element can give stronger insertions with stronger phenotypes. Increased copy number of the transgene by recombination with other transgenic lines containing alternate insertion sites can also give stronger phenotypes. Progeny from this cross with novel/multiple insertions are selected based on a change in eye color. Flies carrying higher copy numbers or stronger insertions of CTFAPP transgene are then crossed with elav-GAL4 driver strains, and locomotor ability of the progeny is tested in climbing assays. Transgenic lines may exhibit a locomotor phenotype and the flies are classified as strong, medium, weak, or very weak as compared among themselves and to elav-GAL4 driver control flies.

A double transgenic Drosophila carrying CTFAPP and tauwt transgenes is then generated bycrossing or recombination with a tauwt transgenic Drosophila with a CTFAPP transgenic Drosophila. Locomotor ability is assessed and classified as strong, medium, weak, or very weak as compared to elav-GAL4 driver control flies.

Climbing performance as a function of age is determined for populations of flies of various genotypes at about 25° C. Climbing assays are performed in groups of approximately 10 individuals of the same age.

Drosophila brain is then cyrosectioned, and horizontal cross sections of elav-GAL4; pUAS-_{2N4R}tauwt/pExP-UAS-CTFAPP flies are immunostained with anti-tau conformation dependent antibodies ALZ50 and MCI. Positive staining of neurons may be observed with both MCI antibody and ALZ50 antibody. The result shows that tau protein, which is expressed in the brain of elav-GAL4; p UAS-_{2N4R}tauwt/pExP-UAS-CTFAPPdouble transgenic Drosophila, exhibits protein conformations associated with Alzheimer's disease.

Thioflavin-S staining is also performed on cells and neurites of the transgenic flies described herein to assess the presence of amyloid. When stained with Thioflavin-S, amyloids fluoresce under a fluorescence microscope. The methods for Thioflavin-S staining are well known in the art. Flies are developed at about 25° C. Thioflavin-S positive cells are not observed in flies expressing tau only. Thioflavin-S positive cells are observed in flies expressing CTFAPP only. However, the number of Thioflavin-5-positive cells is expected to be greater in flies expressing both tau and CTFAPP.

### Example 3

### Effect of CTFAPP and tau on Neurodegeneration

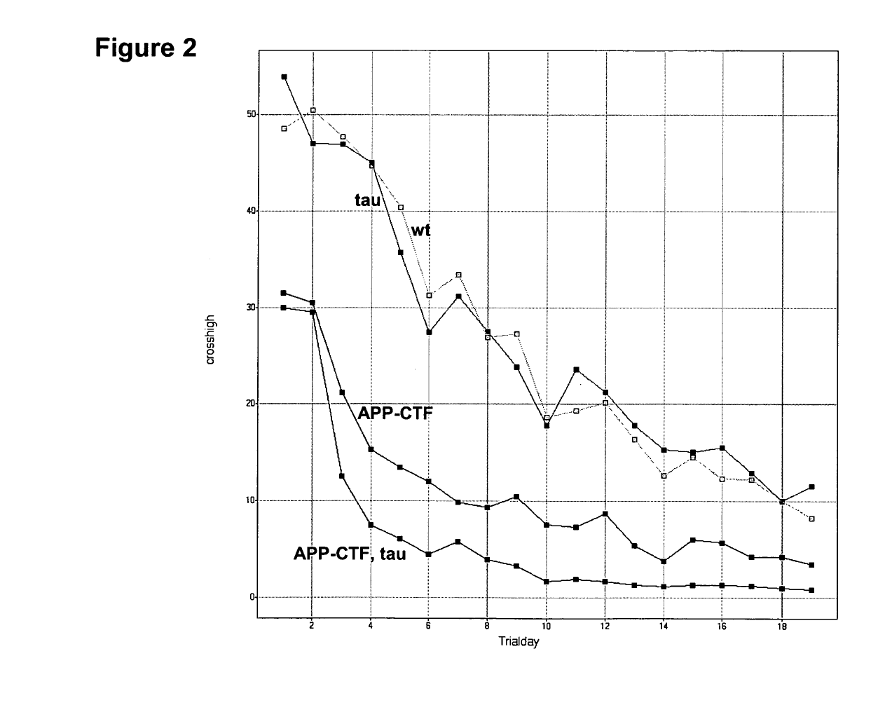

Climbing phenotypes were evaluated for wild type Drosophila as well as Drosophila containing either the CTFAPP or tau transgenes or both transgenes. Transgenes were under control of the elav-GAL4 driver, which results in selective expression of the transgene in the central nervous system. Flies were reared at 25 C. Climbing phenotype was assessed using the “crosshigh” metric described above. The data are shown in FIG. 2. The data indicate that age-dependent neurodegeneration is accelerated by the expression of CTFAPP and further accelerated by the expression of both CTFAPP and tau.

### Example 4

### Screening for a Therapeutic Agent

To screen for a therapeutic agent effective against Alzheimer's disease, candidate agents are administered to a plurality of the elav-GAL4; pUAS-_{2N4R}tauwt/pExP-UAS-CTFAPP-transgenic fly larvae that carry the GMR-GAL4 driver and the transgenes pExP-UAS-CTFAPP-fin combination with pUAS-_{2N4R}tauwt. Candidate agents are microinjected into third instar transgenic Drosophila melanogaster larvae (three to five day old larvae). Larvae are injected through the cuticle into the hemolymph with defined amounts of each compound using a hypodermic needle. Following injection, the larvae are placed into vials for completion of their development. After eclosion, the adult flies are anesthetized with CO₂ and visually inspected utilizing a dissecting microscope to assess for the reversion of the Drosophila eye phenotype as compared to control flies in which a candidate agent was not administered. An observed reversion of the elav-GAL4; pUAS-_{2N4R}tauwt/pExP-UAS-CTFAPP-I transgenic fly eye phenotype towards the phenotype displayed by the control GMR-GAL4 driver strain is indicative of an agent that is useful for the treatment of AD.

1. A transgenic Drosophila whose genome comprises a first DNA sequence shown in SEQ ID NO:1, a second DNA sequence shown in SEQ ID NO:16, and a third DNA sequence encoding Gal4, wherein said first DNA sequence is fused to a DNA sequence shown in SEQ ID NO:5 and said third DNA sequence is operatively linked to an elav promoter.

2. A transgenic fly whose genome comprises a first DNA sequence that encodes a carboxy terminal fragment of human amyloid precursor protein, and a second DNA sequence that encodes a tau protein, wherein each of said first and second DNA sequences is operatively linked to an expression control sequence.

3. The transgenic fly of claim 2, wherein the second DNA sequence encodes a polypeptide comprising the amino acid sequence of a human tau protein.

4. The transgenic fly of claim 2 which is Drosophila.

5. The transgenic fly of claim 2, wherein the expression control sequence linked to either the first or second DNA sequence is tissue specific.

6. The transgenic fly of claim 5, wherein the expression control sequence linked to either the first or second DNA sequence comprises a UAS control element, wherein said fly further comprises a third DNA sequence encoding Gal4, and wherein the third DNA sequence is operatively linked to a tissue-specific promoter or enhancer.

7. The transgenic fly of claim 6, wherein said promoter or enhancer is specific for pan-neural expression.

8. The transgenic fly of claim 6, wherein said promoter or enhancer is specific for expression in eye or central nervous system.

9. The transgenic fly of claim 6, wherein said promoter or enhancer is selected from the group consisting of elav, sca, Nrv2, Dmef2, Cha, TH, P, CaMKII, GMR, OK107, C164, wingless, vestigial, sevenless, eyeless, and gcm.

10. The transgenic fly of claim 2, wherein the first DNA sequence is fused to a DNA sequence encoding a signal peptide.

11. The transgenic fly of claim 10, wherein the signal peptide is from a protein selected from the group consisting of human APP, APPL, wg, aos, presenilin, windbeutel, and Vinc.

12. The transgenic fly of claim 2 which is in an embryonic, larval, pupal, or adult stage.

13. The transgenic fly of claim 2 which has an altered phenotype.

14. The transgenic fly of claim 13, wherein the altered phenotype is selected from the group consisting of a locomotor dysfunction, a behavioural phenotype, a morphological phenotype, and a biochemical phenotype.

15. The transgenic fly of claim 2, wherein the carboxy terminal fragment of human amyloid precursor protein comprises the intracellular domain, the transmembrane domain, and a portion of the extracellular domain of human amyloid precursor protein.

16. A primary cell culture prepared from the transgenic fly of claim 2.

17. A transgenic fly whose genome comprises a DNA sequence that encodes a mutant carboxy terminal fragment of human amyloid precursor protein, wherein the DNA sequence is operatively linked to an expression control sequence, and wherein the mutant carboxy terminal fragment of human amyloid precursor protein is not the London mutant.

18. A primary cell culture prepared from the transgenic fly of claim 17.

19. A method for identifying an agent active in neurodegenerative disease, comprising the steps of:
(a) contacting a candidate agent with the transgenic fly of claim 2; and
(b) observing a selected phenotype of the transgenic fly;
wherein a difference in the observed phenotype between the transgenic fly contacted with the candidate agent and a control transgenic fly not contacted with the candidate agent is indicative of an agent active in neurodegenerative disease.

20. The method of claim 19, wherein the transgenic fly is Drosophila.

21. The method of claim 19, wherein the transgenic fly is in an embryonic, larval, pupal, or adult stage.

22. The method of claim 19, wherein the expression control sequence is tissue specific.

23. The method of claim 19, wherein the expression control sequence comprises a UAS control element, wherein said fly further comprises a third DNA sequence encoding GAL44, and wherein the third DNA sequence is operatively linked to a tissue-specific promoter or enhancer.

24. The method of claim 23, wherein said promoter or enhancer is specific for pan-neural expression.

25. The method of claim 23, wherein said promoter or enhancer is specific for expression in eye or central nervous system.

26. The method of claim 23, wherein said promoter or enhancer is selected from the group consisting of elav, sca, Nrv2, Dmef2, Cha, TH, P, CaMKII, GMR, OK107, C164, wingless, vestigial, sevenless, eyeless, and gcm.

27. The method of claim 23, wherein the first DNA sequence of the transgenic fly is fused to a sequence encoding a signal peptide.

28. The method of claim 23, wherein the phenotype is selected from the group consisting of a locomotor disjunction, a behavioural phenotype, a morphological phenotype, and a biochemical phenotype.

29. A method for identifying an agent active in neurodegenerative disease, comprising the steps of:
(a) contacting a candidate agent with the transgenic fly of claim 1 and with a control wild type fly; and
(b) observing a selected phenotype in the transgenic fly and the control fly;
wherein a difference in the observed phenotype between the transgenic fly and the control fly is indicative of an agent active in neurodegenerative disease.

30. A method for identifying an agent active in neurodegenerative disease, comprising the steps of:
(a) contacting a candidate agent with a transgenic cell from the primary cell culture of claim 16 and with a control cell from a culture prepared from a wild type fly; and
(b) observing a selected phenotype in the transgenic cell and the control cell;
wherein a difference in the observed phenotype between the transgenic cell and the control cell is indicative of an agent active in neurodegenerative disease.

31. A method for identifying an agent active in neurodegenerative disease, comprising the steps of:
(a) contacting a candidate agent with a transgenic cell from the primary cell culture of claim 16; and
(b) observing a selected phenotype in the transgenic cell;
wherein a difference in the observed phenotype between the transgenic cell contacted with the candidate agent and a transgenic cell not contacted with the candidate agent is indicative of an agent active in neurodegenerative disease.

32. The method of claim 30 or claim 31, wherein the phenotype is selected from the group consisting of cell morphology, the aggregation state of the cell, the presence or appearance of intracellular microfibrillary tangles, the presence or appearance of extracellular plaques, the solubility of an amyloid polypeptide, the phosphorylation state of tau, and sensitivity to oxidative stress.

33. A method of identifying a gene which can affect Alzheimer's disease, comprising the steps of:
(a) crossing the transgenic fly of claim 2 or the transgenic fly of claim 24 with a fly whose genome comprises a mutation in a selected gene; and
(b) observing the progeny that possess the transgenes of the fly of claim 2 or the transgene of the fly of claim 17 and the selected gene for alteration of a phenotype associated with said transgenes of the fly of claim 2 or said transgene of the fly of claim 16;
wherein alteration of said phenotype indicates that the selected gene can affect Alzheimer's disease.


SEQUENCE LISTING (18 sequences)

SEQ ID NO: 1 - DNA, 297 bp, Homo sapiens
gacgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaac

SEQ ID NO: 2 - protein, 99 aa, Homo sapiens
DAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 3 - DNA, 300 bp, Homo sapiens
atggacgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaac

SEQ ID NO: 4 - protein, 100 aa, Homo sapiens
MDAEFRHDSGYEVHHQKLVFFAEDVGSNKGAIIGLMVGGVVIATVIVITLVMLKKKQYTSIHHGVVEVDAAVTPEERHLSKMQQNGYENPTYKFFEQMQN

SEQ ID NO: 5 - protein, 17 aa, Homo sapiens
MLPGLALLLLAAWTARA

SEQ ID NO: 6 - protein, 18 aa, Drosophila melanogaster
MDISYIFVICLMALSGGS

SEQ ID NO: 7 - protein, 24 aa, Drosophila melanogaster
MPTTLMLLPCMLLLLLTAAAVAVG

SEQ ID NO: 8 - protein, 27 aa, Drosophila melanogaster
MCAALRRNLLLRSLWVVLAIGTAQVQA

SEQ ID NO: 9 - protein, 20 aa, Drosophila melanogaster
MAAVNLQASCSSGLASEDDA

SEQ ID NO: 10 - protein, 23 aa, Drosophila melanogaster
MMHILVTLLLVAIHSIPTTWAVT

SEQ ID NO: 11 - DNA, 3747 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagagcctgaaagtggtaaggtggtccaggaaggcttcctccgagagccaggccccccaggtctgagccaccagctcatgtccggcatgcctggggctcccctcctgcctgagggccccagagaggccacacgccaaccttcggggacaggacctgaggacacagagggcggccgccacgcccctgagctgctcaagcaccagcttctaggagacctgcaccaggaggggccgccgctgaagggggcagggggcaaagagaggccggggagcaaggaggaggtggatgaagaccgcgacgtcgatgagtcctccccccaagactcccctccctccaaggcctccccagcccaagatgggcggcctccccagacagccgccagagaagccaccagcatcccaggcttcccagcggagggtgccatccccctccctgtggatttcctctccaaagtttccacagagatcccagcctcagagcccgacgggcccagtgtagggcgggccaaagggcaggatgcccccctggagttcacgtttcacgtggaaatcacacccaacgtgcagaaggagcaggcgcactcggaggagcatttgggaagggctgcatttccaggggcccctggagaggggccagaggcccggggcccctctttgggagaggacacaaaagaggctgaccttccagagccctctgaaaagcagcctgctgctgctccgcgggggaagcccgtcagccgggtccctcaactcaaagctcgcatggtcagtaaaagcaaagacgggactggaagcgatgacaaaaaagccaagacatccacacgttcctctgctaaaaccttgaaaaataggccttgccttagccccaaactccccactcctggtagctcagaccctctgatccaaccctccagccctgctgtgtgcccagagccaccttcctctcctaaacacgtctcttctgtcacttcccgaactggcagttctggagcaaaggagatgaaactcaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 12 - DNA, 2796 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 13 - DNA, 2622 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 14 - DNA, 2529 bp, Homo sapiens
cctcccctggggaggctcgcgttcccgctgctcgcgcctgccgcccgccggcctcaggaacgcgccctctcgccgcgcgcgccctcgcagtcaccgccacccaccagctccggcaccaacagcagcgccgctgccaccgcccaccttctgccgccgccaccacagccaccttctcctcctccgctgtcctctcccgtcctcgcctctgtcgactatcaggtgaactttgaaccaggatggctgagccccgccaggagttcgaagtgatggaagatcacgctgggacgtacgggttgggggacaggaaagatcaggggggctacaccatgcaccaagaccaagagggtgacacggacgctggcctgaaagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgcccccagctgctcctcgcagttcggttaattggttaatcacttaacctgcttttgtcactcggctttggctcgggacttcaaaatcagtgatgggagtaagagcaaatttcatctttccaaattgatgggtgggctagtaataaaatatttaaaaaaaaacattcaaaaacatggccacatccaacatttcctcaggcaattccttttgattcttttttcttccccctccatgtagaagagggagaaggagaggctctgaaagctgcttctgggggatttcaagggactgggggtgccaaccacctctggccctgttgtgggggttgtcacagaggcagtggcagcaacaaaggatttgaaaactttggtgtgttcgtggagccacaggcagacgatgtcaaccttgtgtgagtgtgacgggggttggggtggggcgggaggccacgggggaggccgaggcaggggctgggcagaggggaggaggaagcacaagaagtgggagtgggagaggaagccacgtgctggagagtagacatccccctccttgccgctgggagagccaaggcctatgccacctgcagcgtctgagcggccgcctgtccttggtggccgggggtgggggcctgctgtgggtcagtgtgccaccctctgcagggcagcctgtgggagaagggacagcgggttaaaaagagaaggcaagcctggcaggagggttggcacttcgatgatgacctccttagaaagactgaccttgatgtcttgagagcgctggcctcttcctccctccctgcagggtagggcgcctgagcctaggcggttccctctgctccacagaaaccctgttttattgagttctgaaggttggaactgctgccatgattttggccactttgcagacctgggactttagggctaaccagttctctttgtaaggacttgtgcctcttgggagacgtccacccgtttccaagcctgggccactggcatctctggagtgtgtgggggtctgggaggcaggtcccgagccccctgtccttcccacggccactgcagtcaccccgtctgcgccgctgtgctgttgtctgccgtgagagcccaatcactgcctatacccctcatcacacgtcacaatgtcccgaattc

SEQ ID NO: 15 - protein, 414 aa, Homo sapiens [sequence omitted]

SEQ ID NO: 16 - DNA, 1421 bp, Homo sapiens
atggctgagccccgccaggagttcgaagtgatggaagatcacgctgggacgtacgggttgggggacaggaaagatcaggggggctacaccatgcaccaagaccaagagggtgacacggacgctggcctgaaagaatctcccctgcagacccccactgaggacggatctgaggaaccgggctctgaaacctctgatgctaagagcactccaacagcggaagatgtgacagcacccttagtggatgagggagctcccggcaagcaggctgccgcgcagccccacacggagatcccagaaggaaccacagctgaagaagcaggcattggagacacccccagcctggaagacgaagctgctggtcacgtgacccaagctcgcatggtcagtaaaagcaaagacgggactggaagcgatgacaaaaaagccaagggggctgatggtaaaacgaagatcgccacaccgcggggagcagcccctccaggccagaagggccaggccaacgccaccaggattccagcaaaaaccccgcccgctccaaagacaccacccagctctggtgaacctccaaaatcaggggatcgcagcggctacagcagccccggctccccaggcactcccggcagccgctcccgcaccccgtcccttccaaccccacccacccgggagcccaagaaggtggcagtggtccgtactccacccaagtcgccgtcttccgccaagagccgcctgcagacagcccccgtgcccatgccagacctgaagaatgtcaagtccaagatcggctccactgagaacctgaagcaccagccgggaggcgggaaggtgcagataattaataagaagctggatcttagcaacgtccagtccaagtgtggctcaaaggataatatcaaacacgtcccgggaggcggcagtgtgcaaatagtctacaaaccagttgacctgagcaaggtgacctccaagtgtggctcattaggcaacatccatcataaaccaggaggtggccaggtggaagtaaaatctgagaagcttgacttcaaggacagagtccagtcgaagattgggtccctggacaatatcacccacgtccctggcggaggaaataaaaagattgaaacccacaagctgaccttccgcgagaacgccaaagccaagacagaccacggggcggagatcgtgtacaagtcgccagtggtgtctggggacacgtctccacggcatctcagcaatgtctcctccaccggcagcatcgacatggtagactcgccccagctcgccacgctagctgacgaggtgtctgcctccctggccaagcagggtttgtgatcaggcccctggggcggtcaataattgtggagaggagagaatgagagagtgtggaaaaaaaaagaataatgacccggcccccgccctctgccccc

SEQ ID NO: 17 - DNA, 9375 bp, Artificial
  pExp-UAS APP-CTFI vector
gtctggccattctcatcgtgagcttccgggtgctcgcatatctggctctaagacttcgggcccgacgcaaggagtagccgacatatatccgaaataactgcttgtttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggcccatgatgaaataacataaggtggtcccgtcgaaagccgaagcttaccgaagtatacacttaaattcagtgcacgtttgcttgttgagaggaaaggttgtgtgcggacgaatttttttttgaaaacattaacccttacgtggaataaaaaaaaatgaaatattgcaaattttgctgcaaagctgtgactggagtaaaattaattcacgtgccgaagtgtgctattaagagaaaattgtgggagcagagccttgggtgcagccttggtgaaaactcccaaatttgtgatacccactttaatgattcgcagtggaaggctgcacctgcaaaaggtcagacatttaaaaggaggcgactcaacgcagatgccgtacctagtaaagtgatagagcctgaaccagaaaagataaaagaaggctataccagtgggagtacacaaacagagtaagtttgaatagtaaaaaaaatcatttatgtaaacaataacgtgactgtgcgttaggtcctgttcattgtttaatgaaaataagagcttgagggaaaaaattcgtactttggagtacgaaatgcgtcgtttagagcagcagccgaattaattctagttccagtgaaatccaagcattttctaaattaaatgtattcttattattatagttgttatttttgatatatataaacaacactattatgcccaccatttttttgagatgcatctacacaaggaacaaacactggatgtcactttcagttcaaattgtaacgctaatcactccgaacaggtcacaaaaaattaccttaaaaagtcataatattaaattagaataaatatagctgtgagggaaatatatacaaatatattggagcaaataaattgtacatacaaatatttattactaatttctattgagacgaaatgaaccactcggaaccatttgagcgaaccgaatcgcgcggaactaacgacagtcgctccaaggtcgtcgaacaaaaggtgaatgtgttgcggagagcgggtgggagacagcgaaagagcaactacgaaacgtggtgtggtggaggtgaattatgaagagggcgcgcgatttgaaaagtatgtatataaaaaatatatcccggtgttttatgtagcgataaacgagtttttgatgtaaggtatgcaggtgtgtaagtcttttggttagaagacaaatccaaagtctacttgtggggatgttcgaaggggaaatacttgtattctataggtcatatcttgtttttattggcacaaatataattacattagctttttgagggggcaataaacagtaaacacgatggtaataatggtaaaaaaaaaaaacaagcagttatttcggatatatgtcggctactccttgcgtcgggcccgaagtcttagagccagatatgcgagcacccggaagctcacgatgagaatggccagacccacgtagtccagcggcagatcggcggcggagaagttaagcgtctccaggatgaccttgcccgaactggggcacgtggtgttcgacgatgtgcagctaatttcgcccggctccacgtccgcccattggttaatcagcagaccctcgttggcgtaacggaaccatgagaggtacgacaaccatttgaggtatactggcaccgagcccgagttcaagaagaagccgccaaagagcaggaatggtatgataaccggcggacccacagacagcgccatcgaggtcgaggagctggcgcaggatattagatatccgaaggacgttgacacattggccaccagagtgaccagcgccaggcagttgaagaagtgcagcactccggcccgcagtccgatcatcggataggcaatcgccgtgaagaccagtggcactgtgagaaaaagcggcaattcggcaatcgttttgcccagaaagtatgtgtcacagcgataaagtcgacttcgggcctccctcataaaaactggcagctctgaggtgaacacctaaatcgaatcgattcattagaaagttagtaaattattgaaatgcaaatgtattctaaacatgacttacatttatcgtggcaaagacgttttgaaaggtcatgttggtcaggaagaggaagatggctccgttgatattcatcacacccacttgcgtgagttgttggcccaaaaagatgaggccaatcaagatggcaaccatctgcaaattaaaatgttactcgcatctcattaatattcgcgagttaaatgaaatttatttatcttctgcaaaactataaactatacatctcattgaaaaaaactaagaagggtgtggaatcaggcaattctatctaaaatctagcgaatttgtttccaagaattgtaagcgttatatcatttgtttccactggaaccactcaccgttgtctgaataagtcgcacttttacgaggagtggttccttgagcaccgacagccaggatcgccacaggaccgcccggaactgcatgaaccaggtggccttgtaggtgtacccattctccggctgctccagtggcttctccagatttttggtggccaacaactgctccatatcccgggctactttgctaatggcaaaattgtcgccatatcttggcgatccgatcacgggactcgatctcccgtccgggcacaacggccaacacctgtacgtaaaagtccgccggattgtagttggtaggacactgggcacccacgctggataggagttgagatgtaatgtaatgctagatacccttaataaacacatcgaactcactaggaaaagaagtcgacggcttcgctgggagtgcccaagaaagctaccctgccctcggccatcagaaggatcttgtcaaagagctcaaacagctcggaagacggctgatgaatggtcaggatgacggtcttgcccttctgcgacagcttcttcagcacctggacgacgctgtgggcggtaaatgagtccagtccggaggtgggctcatcgcagatcagaagcggcggatcggttagtgcctcggaggcgaatgccagacgcttcctttctccgccggacagacctttcaccctgccgggcacaccgatgatcgtgtgctgacatttgctgagcgaaagctcctggatcacctgatccacgcgggccactcgctgccgataggtcagatgtcgtggcatccgcaccatggcttggaaaatcaggtgttccctggccgttagggagccgataaagaggtcatcctgctggacataggcgcacctggcctgcatctccttggcgtccacaggttggccattgagcagtcgcatcccggatggcgatacttggatgccctgcggcgatcgaaaggcaagggcattcagcagggtcgtctttccggcaccggaactgcccatcacggccaaaagttcgcccggataggccacgccgcaaactgagtttcaaattggtaattggaccctttattaagatttcacacagatcagccgactgcgaatagaaactcaccgttcttgagcaaatgtttcctgggcgccggtatgtgtcgctcgttgcagaatagtccgcgtgtccggttgaccagctgccgccatccggagcccggctgattgaccgccccaaagatgtccatattgtgccaggcataggtgaggttctcggctagttggccgctccctgaaccggagtcctccggcggactgggtggcaggagcgtgccgtagtttttggcctgcccgaagccctggttaatgcagctctgcgaagcgtccgctgtcaccctgcaatgataggggatctcaaatatcaactacaagcgttatgctcatctaaccccgaacaaaacgaagtatcctacgaagtaggtttatacttttatttattttttgtgcatagcttaaaatatctggttgttatattttttgtaaaaaagaatgtagtcgaaaatgaatgcctttagatgtcttgatcatgatatgatcttaaaaattgtcttatatagcgagcacagctaccagaataatctgtttcgtgtcactatttgtttgtgcgattgcggtttgggatttttgtgggtcgcagttctcacgccgcagacaatttgatgttgcaatcgcagttcctatagatcaagtgaacttaagatgtatgcacatgtactactcacattgttcagatgctcggcagatgggtgtttgctgcctccgcgaattaatagctcctgatcctcttggcccattgccgggatttttcacactttcccctgcttacccacccaaaaccaatcaccaccccaatcactcaaaaaacaaacaaaaataagaagcgagaggagttttggcacagcactttgtgtttaattgatggcgtaaaccgcttggagcttcgtcacgaaaccgctgacaaagtgcaactgaaggcggacattgacgctaggtaacgctacaaacggtggcgaaagagatagcggacgcagcggcgaaagagacggcgatatttctgtggacagagaaggaggcaaacagcgctgactttgagtggaatgtcattttgagtgagaggtaatcgaaagaacctggtacatcaaatacccttggatcgaagtaaatttaaaactgatcagataagttcaatgatatccagtgcagtaaaaaaaaaaaatgttttttttatctactttccgcaaaaatgggttttattaacttacatacatactagaattaattcctcgagtaggccggccatggtaccgaggatccaagcttgcatgcctgcaggtcggagtactgtcctccgagcggagtactgtcctccgagcggagtactgtcctccgagcggagtactgtcctccgagcggagtactgtcctccgagcggagactctagcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagggaattgggaattctaggcgcgccaatcaacatgctgcccggtctggccctgctgctgctggccgcctggaccgctcgtgccgatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaacgagcagaagctgatctccgaggaggacctgtaaccgcggtctagaaggactaagcgtcgcgccacttcaacgctcgatgggagcgtcattggtgggcggggtaaccgtcgaaatcagtgtttacgcttccaatcgcaacaaaaaattcactgcaacactgaaaagcatacgaaaacgatgaagattgtacgagaaaccataaagtattttatccacaaagacacgtatagcagaaaagccaagttaactcggcgataagttgtgtacacaagaataaaatcggccagattcagtgttgtcagaaataagaaaaccccactatgtttttctttgccttttctttctcccagcgatcattcatttcgtggtgaaagaacggggtcattgcacggagtttcgactgcgggaaagcagagctgccgttcacttcgtctataattagcgctttctattttccccgattcgggccgctgctgcgcttttccgcctgctgtttgtggcaagtgtagcagcaggctgtgcacgcaggtggcatgcacttggctttccaccgttggtatcgattctctgggacgatgagtcattcctttcggggccacagcataatcgttgccagctcaccgaaatggtgacttcatttcttaactgccgtcaagcatgcgattgtacatacatacatatttatatatgtacatatttatgtgactatggtaggtcgatataatagcaatcaacgcaagcaaatgtgtcagtcctgcttacaggaacgattctatttagtaattttcgttgtataaagtaattatgtatgtatgtaagccccataaatctgaaacaattaggcaaaaccatgcgaagctcgcactagtgcctgcagccaagctttgcgtactcgcaaattattaaaaataaaactttaaaaataatttcgtctaattaatattatgagttaattcaaaccccacggacatgctaagggttaatcaacaatcatatcgctgtctcactcagactcaatacgacactcagaatactattcctttcactcgcacttattgcaagcatacgttaagtggatgtctcttgccgacgggaccaccttatgttatttcatcatg

SEQ ID NO: 18 - DNA, 9462 bp, Artificial
  pExP-UAS APP-CTFII vector
gtctggccattctcatcgtgagcttccgggtgctcgcatatctggctctaagacttcgggcccgacgcaaggagtagccgacatatatccgaaataactgcttgtttttttttttaccattattaccatcgtgtttactgtttattgccccctcaaaaagctaatgtaattatatttgtgccaataaaaacaagatatgacctatagaatacaagtatttccccttcgaacatccccacaagtagactttggatttgtcttctaaccaaaagacttacacacctgcataccttacatcaaaaactcgtttatcgctacataaaacaccgggatatattttttatatacatacttttcaaatcgcgcgccctcttcataattcacctccaccacaccacgtttcgtagttgctctttcgctgtctcccacccgctctccgcaacacattcaccttttgttcgacgaccttggagcgactgtcgttagttccgcgcgattcggtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccttcttcttgaactcgggctcggtgccagtatacctcaaatggttgtcgtacctctcatggttccgttacgccaacgagggtctgctgattaaccaatgggcggacgtggagccgggcgaaattagctgcacatcgtcgaacaccacgtgccccagttcgggcaaggtcatcctggagacgcttaacttctccgccgccgatctgccgctggactacgtgggtctggcccatgatgaaataacataaggtggtcccgtcgaaagccgaagcttaccgaagtatacacttaaattcagtgcacgtttgcttgttgagaggaaaggttgtgtgcggacgaatttttttttgaaaacattaacccttacgtggaataaaaaaaaatgaaatattgcaaattttgctgcaaagctgtgactggagtaaaattaattcacgtgccgaagtgtgctattaagagaaaattgtgggagcagagccttgggtgcagccttggtgaaaactcccaaatttgtgatacccactttaatgattcgcagtggaaggctgcacctgcaaaaggtcagacatttaaaaggaggcgactcaacgcagatgccgtacctagtaaagtgatagagcctgaaccagaaaagataaaagaaggctataccagtgggagtacacaaacagagtaagtttgaatagtaaaaaaaatcatttatgtaaacaataacgtgactgtgcgttaggtcctgttcattgtttaatgaaaataagagcttgagggaaaaaattcgtactttggagtacgaaatgcgtcgtttagagcagcagccgaattaattctagttccagtgaaatccaagcattttctaaattaaatgtattcttattattatagttgttatttttgatatatataaacaacactattatgcccaccatttttttgagatgcatctacacaaggaacaaacactggatgtcactttcagttcaaattgtaacgctaatcactccgaacaggtcacaaaaaattaccttaaaaagtcataatattaaattagaataaatatagctgtgagggaaatatatacaaatatattggagcaaataaattgtacatacaaatatttattactaatttctattgagacgaaatgaaccactcggaaccatttgagcgaaccgaatcgcgcggaactaacgacagtcgctccaaggtcgtcgaacaaaaggtgaatgtgttgcggagagcgggtgggagacagcgaaagagcaactacgaaacgtggtgtggtggaggtgaattatgaagagggcgcgcgatttgaaaagtatgtatataaaaaatatatcccggtgttttatgtagcgataaacgagtttttgatgtaaggtatgcaggtgtgtaagtcttttggttagaagacaaatccaaagtctacttgtggggatgttcgaaggggaaatacttgtattctataggtcatatcttgtttttattggcacaaatataattacattagctttttgagggggcaataaacagtaaacacgatggtaataatggtaaaaaaaaaaaacaagcagttatttcggatatatgtcggctactccttgcgtcgggcccgaagtcttagagccagatatgcgagcacccggaagctcacgatgagaatggccagacccacgtagtccagcggcagatcggcggcggagaagttaagcgtctccaggatgaccttgcccgaactggggcacgtggtgttcgacgatgtgcagctaatttcgcccggctccacgtccgcccattggttaatcagcagaccctcgttggcgtaacggaaccatgagaggtacgacaaccatttgaggtatactggcaccgagcccgagttcaagaagaagccgccaaagagcaggaatggtatgataaccggcggacccacagacagcgccatcgaggtcgaggagctggcgcaggatattagatatccgaaggacgttgacacattggccaccagagtgaccagcgccaggcagttgaagaagtgcagcactccggcccgcagtccgatcatcggataggcaatcgccgtgaagaccagtggcactgtgagaaaaagcggcaattcggcaatcgttttgcccagaaagtatgtgtcacagcgataaagtcgacttcgggcctccctcataaaaactggcagctctgaggtgaacacctaaatcgaatcgattcattagaaagttagtaaattattgaaatgcaaatgtattctaaacatgacttacatttatcgtggcaaagacgttttgaaaggtcatgttggtcaggaagaggaagatggctccgttgatattcatcacacccacttgcgtgagttgttggcccaaaaagatgaggccaatcaagatggcaaccatctgcaaattaaaatgttactcgcatctcattaatattcgcgagttaaatgaaatttatttatcttctgcaaaactataaactatacatctcattgaaaaaaactaagaagggtgtggaatcaggcaattctatctaaaatctagcgaatttgtttccaagaattgtaagcgttatatcatttgtttccactggaaccactcaccgttgtctgaataagtcgcacttttacgaggagtggttccttgagcaccgacagccaggatcgccacaggaccgcccggaactgcatgaaccaggtggccttgtaggtgtacccattctccggctgctccagtggcttctccagatttttggtggccaacaactgctccatatcccgggctactttgctaatggcaaaattgtcgccatatcttggcgatccgatcacgggactcgatctcccgtccgggcacaacggccaacacctgtacgtaaaagtccgccggattgtagttggtaggacactgggcacccacgctggataggagttgagatgtaatgtaatgctagatacccttaataaacacatcgaactcactaggaaaagaagtcgacggcttcgctgggagtgcccaagaaagctaccctgccctcggccatcagaaggatcttgtcaaagagctcaaacagctcggaagacggctgatgaatggtcaggatgacggtcttgcccttctgcgacagcttcttcagcacctggacgacgctgtgggcggtaaatgagtccagtccggaggtgggctcatcgcagatcagaagcggcggatcggttagtgcctcggaggcgaatgccagacgcttcctttctccgccggacagacctttcaccctgccgggcacaccgatgatcgtgtgctgacatttgctgagcgaaagctcctggatcacctgatccacgcgggccactcgctgccgataggtcagatgtcgtggcatccgcaccatggcttggaaaatcaggtgttccctggccgttagggagccgataaagaggtcatcctgctggacataggcgcacctggcctgcatctccttggcgtccacaggttggccattgagcagtcgcatcccggatggcgatacttggatgccctgcggcgatcgaaaggcaagggcattcagcagggtcgtctttccggcaccggaactgcccatcacggccaaaagttcgcccggataggccacgccgcaaactgagtttcaaattggtaattggaccctttattaagatttcacacagatcagccgactgcgaatagaaactcaccgttcttgagcaaatgtttcctgggcgccggtatgtgtcgctcgttgcagaatagtccgcgtgtccggttgaccagctgccgccatccggagcccggctgattgaccgccccaaagatgtccatattgtgccaggcataggtgaggttctcggctagttggccgctccctgaaccggagtcctccggcggactgggtggcaggagcgtgccgtagtttttggcctgcccgaagccctggttaatgcagctctgcgaagcgtccgctgtcaccctgcaatgataggggatctcaaatatcaactacaagcgttatgctcatctaaccccgaacaaaacgaagtatcctacgaagtaggtttatacttttatttattttttgtgcatagcttaaaatatctggttgttatattttttgtaaaaaagaatgtagtcgaaaatgaatgcctttagatgtcttgatcatgatatgatcttaaaaattgtcttatatagcgagcacagctaccagaataatctgtttcgtgtcactatttgtttgtgcgattgcggtttgggatttttgtgggtcgcagttctcacgccgcagacaatttgatgttgcaatcgcagttcctatagatcaagtgaacttaagatgtatgcacatgtactactcacattgttcagatgctcggcagatgggtgtttgctgcctccgcgaattaatagctcctgatcctcttggcccattgccgggatttttcacactttcccctgcttacccacccaaaaccaatcaccaccccaatcactcaaaaaacaaacaaaaataagaagcgagaggagttttggcacagcactttgtgtttaattgatggcgtaaaccgcttggagcttcgtcacgaaaccgctgacaaagtgcaactgaaggcggacattgacgctaggtaacgctacaaacggtggcgaaagagatagcggacgcagcggcgaaagagacggcgatatttctgtggacagagaaggaggcaaacagcgctgactttgagtggaatgtcattttgagtgagaggtaatcgaaagaacctggtacatcaaatacccttggatcgaagtaaatttaaaactgatcagataagttcaatgatatccagtgcagtaaaaaaaaaaaatgttttttttatctactttccgcaaaaatgggttttattaacttacatacatactagaattaattcctcgagtaggccggccatggtaccgaggatccaagcttgcatgcctgcaggtcggagtactgtcctccgagcggagtactgtcctccgagcggagtactgtcctccgagcggagtactgtcctccgagcggagtactgtcctccgagcggagactctagcgagcgccggagtataaatagaggcgcttcgtctacggagcgacaattcaattcaaacaagcaaagtgaacacgtcgctaagcgaaagctaagcaaataaacaagcgcagctgaacaagctaaacaatctgcagtaaagtgcaagttaaagtgaatcaattaaaagtaaccagcaaccaagtaaatcaactgcaactactgaaatctgccaagaagtaattattgaatacaagaagagaactctgaatagggaattgggaattctaggcgcgccaatcaacatgttcaagttcgtcatgatcctggcagtggtgggcgtggccacggctgatgcagaattccgacatgactcaggatatgaagttcatcatcaaaaattggtgttctttgcagaagatgtgggttcaaacaaaggtgcaatcattggactcatggtgggcggtgttgtcatagcgacagtgatcgtcatcaccttggtgatgctgaagaagaaacagtacacatccattcatcatggtgtggtggaggttgacgccgctgtcaccccagaggagcgccacctgtccaagatgcagcagaacggctacgaaaatccaacctacaagttctttgagcagatgcagaactgcggccgcatcttttacccatacgatgttcctgactatgcgggctatccctatgacgtcccggactatgcaggatcctatccatatgacgttccagattacgctgctcagtgcggccgctaaccgcggtctagaaggactaagcgtcgcgccacttcaacgctcgatgggagcgtcattggtgggcggggtaaccgtcgaaatcagtgtttacgcttccaatcgcaacaaaaaattcactgcaacactgaaaagcatacgaaaacgatgaagattgtacgagaaaccataaagtattttatccacaaagacacgtatagcagaaaagccaagttaactcggcgataagttgtgtacacaagaataaaatcggccagattcagtgttgtcagaaataagaaaaccccactatgtttttctttgccttttctttctcccagcgatcattcatttcgtggtgaaagaacggggtcattgcacggagtttcgactgcgggaaagcagagctgccgttcacttcgtctataattagcgctttctattttccccgattcgggccgctgctgcgcttttccgcctgctgtttgtggcaagtgtagcagcaggctgtgcacgcaggtggcatgcacttggctttccaccgttggtatcgattctctgggacgatgagtcattcctttcggggccacagcataatcgttgccagctcaccgaaatggtgacttcatttcttaactgccgtcaagcatgcgattgtacatacatacatatttatatatgtacatatttatgtgactatggtaggtcgatataatagcaatcaacgcaagcaaatgtgtcagtcctgcttacaggaacgattctatttagtaattttcgttgtataaagtaattatgtatgtatgtaagccccataaatctgaaacaattaggcaaaaccatgcgaagctcgcactagtgcctgcagccaagctttgcgtactcgcaaattattaaaaataaaactttaaaaataatttcgtctaattaatattatgagttaattcaaaccccacggacatgctaagggttaatcaacaatcatatcgctgtctcactcagactcaatacgacactcagaatactattcctttcactcgcacttattgcaagcatacgttaagtggatgtctcttgccgacgggaccaccttatgttatttcatcatg